(19) United States
(12) Patent Application Publication
(10) Pub. No.: US 2020/0100856 A1
(43) Pub. Date: Apr. 2, 2020

(54) INFORMATION INPUT APPARATUS AND MEDICAL SYSTEM

(71) Applicant: SONY CORPORATION, TOKYO (JP)

(72) Inventors: KAZUO HONGO, CHIBA (JP); YUKI ITOTANI, TOKYO (JP)

(21) Appl. No.: 16/620,019

(22) PCT Filed: Jun. 4, 2018
(86) PCT No.: PCT/JP2018/021418

§ 371 Date: Dec. 6, 2019

(30) Foreign Application Priority Data
Jun. 15, 2017 (JP) 2017-118029

(51) Int. Cl. A61B 34/37; G06F 3/0346; G06F 3/01; G06F 3/03; A61B 34/00

(52) U.S. Cl. CPC A61B 34/37; G06F 3/0346; A61B 34/76; G06F 3/0312; A61B 34/74; G06F 3/016

(57) ABSTRACT

Provided is an information input apparatus capable of inputting rotation angles of three degrees of freedom. The information input apparatus includes: an outer shell section having a structure of a hollow sphere; a connection section attracting and rotatably supporting the outer shell section; and a rotation detection section configured to detect a rotation angle of the outer shell section. The rotation detection section includes at least one or a combination of two or more of an acceleration sensor, an angle sensor, and a magnetic sensor. The rotation detection section is arranged in a vicinity of a center of the sphere and configured to detect rotation angles of three degrees of freedom of the outer shell section. The outer shell section includes an opening section into which a finger of a user is inserted, and the rotation detection section is configured to detect the rotation angle when the outer shell section is manually operated by the inserted finger.

### TECHNICAL FIELD

The technology disclosed in the present specification relates to an information input apparatus and a medical system capable of inputting rotation angles of three degrees of freedom.

### BACKGROUND ART

Advances in robotics technology have been remarkable in recent years, and the robotics technology has been widely used in workplaces in various industrial fields. In the medical field, for example, master-slave type medical systems have been gradually introduced in endoscopic surgery of the abdomen, thorax, or the like. The endoscopic surgery enables an approach to an affected area without making a large incision in the body of a patient.

In a master-slave system, a user (surgeon) operates a master apparatus including an input user interface (UI), and a remote slave arm traces the movement. In this way, the master-slave system can realize a remote operation of a manipulator. In the case of a medical master-slave system, an end effector of the slave arm is equipped with a medical surgical tool such as forceps or tweezers. The user remotely operates the surgical tool via a master console (see PTL 1, for example).

### CITATION LIST

### Patent Literature

### [PLT 1]

JP 2009-131446A

### SUMMARY

### Technical Problem

An object of the technology disclosed in the present specification is to provide an information input apparatus and a medical system capable of inputting rotation angles of three degrees of freedom.

### Solution to Problem

A first aspect of the technology disclosed in the present specification is an information input apparatus including:

an outer shell section having a structure of a hollow sphere;

a connection section attracting and rotatably supporting the outer shell section; and

a rotation detection section configured to detect a rotation angle of the outer shell section.

The rotation detection section includes at least one or a combination of two or more of an acceleration sensor, an angle sensor, and a magnetic sensor. The rotation detection section is arranged in a vicinity of a center of the sphere and is configured to detect rotation angles of three degrees of freedom of the outer shell section.

A center of gravity of the outer shell section is positioned in a vicinity of a center of the sphere. The outer shell section further includes: a first opening section into which a first finger of a user is inserted; and a second opening section into which a second finger of the user is inserted. Moreover, the information input apparatus further includes a gripping mechanism arranged inside the outer shell section and configured to perform a pinch operation using the first finger and the second finger. Further, the information input apparatus further includes a finger detection sensor configured to detect that the finger has been inserted into the opening section. The finger detection sensor includes an optical sensor or an electrostatic sensor.

Further, a second aspect of the technology disclosed in the present specification is a master-slave type medical system, in which a master apparatus includes:

an operation section including
an outer shell section having a structure of a hollow sphere, and
a rotation detection section configured to detect a rotation angle of the outer shell section; and

a translational structure section attracting and rotatably supporting the outer shell section of the operation section and configured to detect a translation force acting on the outer shell section or present the translation force.

The translational structure section may have a parallel link structure including a plurality of links each supporting the outer shell section at a distal end side and being turnably supported by a main body of the master apparatus at a proximal end side. Further, an actuator configured to rotationally drive each of the plurality of links and present the translation force may be further included.

### Advantageous Effects of Invention

According to the technology disclosed in the present specification, an information input apparatus and a medical system capable of inputting rotation angles of three degrees of freedom can be provided.

It is to be noted that the effects described in the present specification are merely examples, and the effects of the present invention are not limited thereto. Further, the present invention may further provide additional effects other than the effects described above.

Still another object, feature, and advantage of the technology disclosed in the present specification will become clear from further detailed description based on an embodiment described later and the attached drawings.

### BRIEF DESCRIPTION OF DRAWINGS

FIG. 1 is a diagram schematically illustrating an example of a configuration of a master-slave type robot system 1.

FIG. 2 is a view illustrating an external configuration of an information input apparatus 100 capable of inputting rotation angles of three degrees of freedom proposed in the present specification.

FIG. 3 is a view illustrating, as an example, a state of the information input apparatus 100 with an outer shell section 110 rotationally operated.

FIG. 4 is a view illustrating, as an example, a state of the information input apparatus 100 with the outer shell section 110 rotationally operated.

FIG. 5 is a view illustrating a specific example of the configuration of the information input apparatus 100.

FIG. 6 is a view illustrating a specific example of an internal configuration of the information input apparatus 100.

FIG. 7 is a view for describing a gripping operation of a gripping mechanism.

FIG. 8 is another view for describing the gripping operation of the gripping mechanism.

FIG. 9 is further another view for describing the gripping operation of the gripping mechanism.

FIG. 10 is yet another view for describing the gripping operation of the gripping mechanism.

FIG. 11 is a view illustrating an example in which an intermediate member is arranged on a contact surface 121 between the outer shell section 110 and a connection section 120.

FIG. 12 is a view illustrating another application example of the information input apparatus 100.

FIG. 13 is a view illustrating further another application example of the information input apparatus 100.

FIG. 14 is view illustrating a perspective view of a master apparatus 60 to which the information input apparatus 100 is applied as an operation section.

FIG. 15 is a view illustrating a state in which a pair of left and right master apparatuses 60L and 60R is installed.

FIG. 16 is a view illustrating an example of how the master apparatuses 60L and 60R are installed.

FIG. 17 is another view illustrating an example of how the master apparatuses 60L and 60R are installed.

FIG. 18 is a view illustrating a modification of the connection section 120.

FIG. 19 is a view illustrating an example of a configuration of the information input apparatus 100 in which actuators for presenting a rotational reaction force are arranged.

FIG. 20 is a view illustrating the example of the configuration of the information input apparatus 100 in which the actuators for presenting the rotational reaction force are arranged.

### DESCRIPTION OF EMBODIMENT

Hereinafter, an embodiment of the technology disclosed in the present specification will be described in detail with reference to the drawings.

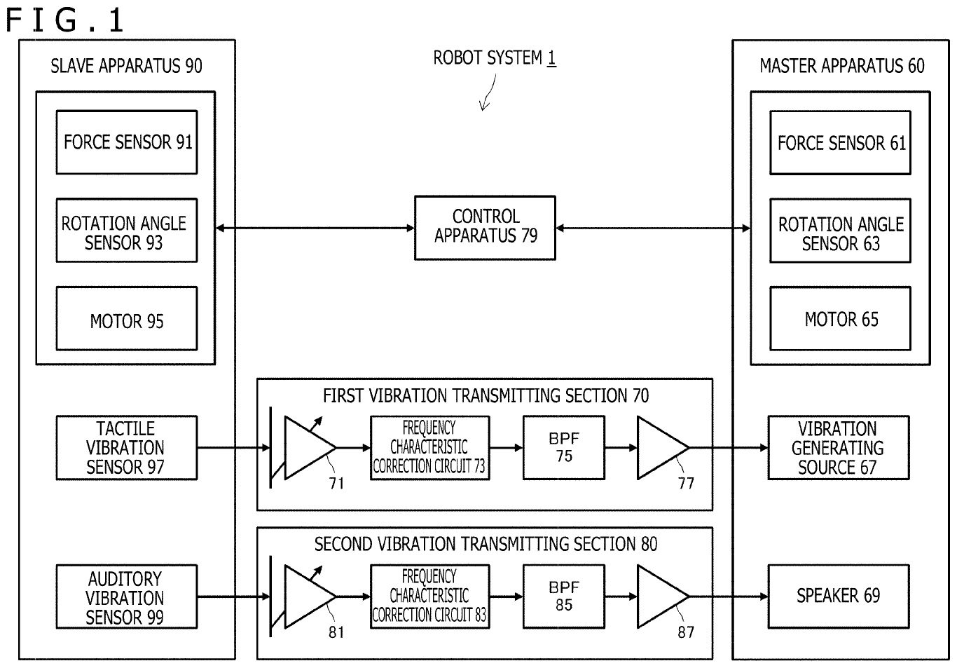

FIG. 1 schematically illustrates an example of a configuration of a master-slave type robot system 1. The illustrated robot system 1 is, for example, a medical robot system that performs endoscopic surgery of the abdomen, thorax, or the like. The robot system 1 includes a master apparatus 60 and a slave apparatus 90. When a user (surgeon) operates the master apparatus 60, an operation command for the slave apparatus 90 is transmitted by wired or wireless communication means, and the slave apparatus 90 is operated.

The slave apparatus 90 is, for example, a forceps unit that includes an arm with multiple degrees of freedom and forceps attached to an end effector of the arm (both the arm and the end effector are omitted in FIG. 1). Further, instead of the forceps, another medical surgical tool such as tweezers or a cutting instrument that touches the patient during surgery, or an imaging apparatus such as an endoscope or a microscope may be attached to the end effector of the arm. The slave apparatus 90 changes the position and orientation of the forceps on the basis of the operation command from the master apparatus 60, and also causes the forceps to perform a gripping operation.

By contrast, the master apparatus 60 is, for example, an arm apparatus that includes an operation section to be operated by the user and an arm with multiple degrees of freedom (both the operation section and the arm are omitted in FIG. 1). The operation section is attached to a distal end of the arm. The user can remotely control the position and attitude of the forceps on the slave apparatus 90 side by displacing the position and attitude of the operation section of the master apparatus 60. Further, the user can remotely operate the gripping operation of the forceps of the slave apparatus 90 by performing the gripping operation on the operation section of the master apparatus 60.

Further, force sense presentation is applied to the robot system 1. The master apparatus 60 presents an external force received by the end effector on the slave apparatus 90 side from an affected area or the like to the user via the operation section or the like. This configuration can contribute to the realization of minimally invasive surgery under the endoscope.

The robot system 1 includes information transmission systems between the master apparatus 60 and the slave apparatus 90: a system for the master apparatus 60 controlling driving of the slave apparatus 90 and presenting a force sense to the user; and a system for transmitting vibration detected on the slave apparatus 90 side to the user. Hereinafter, each information transmission system will be described.

First, the system for controlling the driving of the slave apparatus 90 and presenting a force sense to the user will be described.

In order to realize this information transmission, the robot system 1 further includes a control apparatus 79. The control apparatus 79 drives the slave apparatus 90 according to an instruction input via the master apparatus 60. It is to be noted that, however, part or all of functions of the control apparatus 79 may be included in at least one of the slave apparatus 90 or the master apparatus 60. For example, a CPU (Central Processing Unit) (not illustrated) of at least one of the master apparatus 60 or the slave apparatus 90 functions as the control apparatus 79.

To control driving of the slave apparatus 90, the master apparatus 60 transmits information to the control apparatus 79 when the user operates the operation section attached to the distal end of the arm of the master apparatus 60. This information indicates an instruction to drive the arm of the slave apparatus 90. In a case where a medical surgical tool such as forceps is attached to the end effector of the arm of the slave apparatus 90, the master apparatus 60 may also transmit information indicating an instruction to drive the surgical tool to the control apparatus 79.

The master apparatus 60 includes a force sensor (torque sensor) 61, rotation angle sensors 63, and a motor 65 as constituent elements for controlling driving of the slave apparatus 90 and presenting a force sense. Further, the slave apparatus 90 includes force sensors (torque sensors) 91, rotation angle sensors 93, and motors 95 as constituent elements for controlling driving of the arm of the slave apparatus 90 and presenting a force sense to the master apparatus 60. It is to be noted that a method for controlling driving of the slave apparatus 90 is arbitrary and various known control methods can be applied. Further, since the control apparatus 79 can be configured as appropriate according to a control method employed by the slave apparatus 90, detailed description thereof is omitted.

For example, the force sensor 61 is provided at a connection portion between the arm and the operation section attached to the distal end of the arm on the master apparatus 60 side. The force sensor 61 detects forces acting in directions of three axes orthogonal to each other. That is, the force sensor 61 detects a force input to the operation section by the user. Further, the rotation angle sensors 63 are provided at a plurality of joint portions of the arm and detect the rotation angles of the respective joint portions. The rotation angle sensors 63 may be encoders, for example.

The control apparatus 79 performs various computations related to the control of driving of the slave apparatus 90 on the basis of information input from the force sensor 61 and the rotation angle sensors 63 on the master apparatus 60 side.

For example, in a case where the control apparatus 79 controls the driving of the slave apparatus 90 through the force control, the control apparatus 79 calculates torque to be generated on each motor 95 of the arm of the slave apparatus 90 on the basis of the force acting on the operation section that has been detected by the force sensor 61, and transmits the torque to the slave apparatus 90.

Further, in a case where the control apparatus 79 controls the driving of the slave apparatus 90 through the position control, the control apparatus 79 calculates target values of the rotation angles of the respective joint portions of the arm of the slave apparatus 90 on the basis of the rotation angles of the respective joint portions of the arm that have been detected by the rotation angle sensors 63 on the master apparatus 60 side, and transmits the target values to the slave apparatus 90.

Further, in a case where a surgical tool of the slave apparatus 90 includes a driving portion (for example, in a case where forceps capable of performing the gripping operation are attached to the end effector of the arm), the control apparatus 79 calculates the control amount for driving the surgical tool and transmits the control amount to the slave apparatus 90.

As described above, the control apparatus 79 calculates the control amount for the slave apparatus 90 on the basis of the information input into the master apparatus 60, and transmits a driving signal corresponding to the calculated control amount to the motors 95 on the slave apparatus 90 side. For example, the motors 95 are arranged at the plurality of joint portions of the arm and rotationally drive the respective joint portions.

Moreover, the motors 95 are driven according to the control amount calculated by the control apparatus 79. Accordingly, the arm on the slave apparatus 90 side operates as instructed by the user via (the operation section) of the master apparatus 60. Further, in a case where the end effector of the arm is provided with a surgical tool including a driving portion (for example, forceps capable of performing the gripping operation), the control apparatus 79 transmits a driving signal for a motor for operating this portion, and the motor is driven. Accordingly, the surgical tool is operated as instructed by the user via (the operation section) of the master apparatus 60.

Further, the force sensors 91 detect an external force acting on the surgical tool on the slave apparatus 90 side. For example, the force sensors 91 are provided at the plurality of joint portions of the arm and detect forces (torque) acting on the respective joint portions. Further, for example, the rotation angle sensors 93 are provided at the plurality of joint portions of the arm and detect the rotation angles of the respective joint portions. The rotation angle sensors 93 may be encoders, for example. Information detected by the force sensors 91 and the rotation angle sensors 93 is transmitted to the control apparatus 79. The control apparatus 79 sequentially grasps the current state of the arm on the basis of the information, and calculates the above-described control amount for the slave apparatus 90, taking into account the current state of the arm as well.

Here, each force sensor 91 detects a force acting on the corresponding joint portion. For example, it is assumed that the force acting on the surgical tool attached to the end effector of the arm acts on each joint portion and is detected by the force sensors 91. The control apparatus 79 extracts a component of the force acting on the surgical tool from the forces acting on the respective joint portions that have been detected by the force sensors 91 and calculates the control amount of the motor 65 of the master apparatus 60. The motor 65 includes a servomotor, for example. When the user performs an operation input to the operation section, the control apparatus 79 causes the motor 65 to drive the arm on the master apparatus 60 side so as to give resistance corresponding to the force acting on the surgical tool. In this way, the force acting on the surgical tool can be presented to the user. Therefore, it can be said that the robot system 1 has functions of detecting a force acting on the surgical tool and feeding back the force to the user.

Next, the system for transmitting vibration detected on the slave apparatus 90 side to the user will be described.

In order to realize this information transmission, the robot system 1 further includes a first vibration transmitting section 70 and a second vibration transmitting section 80. The first vibration transmitting section 70 transmits vibration detected by a tactile vibration sensor 97 to the master apparatus 60. The tactile vibration sensor 97 is provided in the slave apparatus 90. Further, the second vibration transmitting section 80 transmits vibration detected by an auditory vibration sensor 99 to the master apparatus 60. The auditory vibration sensor 99 is provided in the slave apparatus 90. The first vibration transmitting section 70 includes an amplifier 71, a frequency characteristic correction circuit 73, a band-pass filter (BPF) 75, and a driving circuit (driver) 77. The second vibration transmitting section 80 includes an amplifier 81, a frequency characteristic correction circuit 83, a band-pass filter (BPF) 85, and a driving circuit (driver) 87. It is to be noted that, however, part or all of the constituent elements of the first vibration transmitting section 70 and the second vibration transmitting section 80 may be provided in at least one of the slave apparatus 90 or the master apparatus 60.

Further, the slave apparatus 90 includes the tactile vibration sensor 97 and the auditory vibration sensor 99 as elements used to transmit vibration to the user. The tactile vibration sensor 97 and the auditory vibration sensor 99 may be attached to a proximal end side of the surgical tool, for example. The tactile vibration sensor 97 detects tactile vibration generated in the surgical tool. The auditory vibration sensor 99 detects auditory vibration (that is, sound) generated in the surgical tool. The tactile vibration sensor 97 includes an acceleration sensor, for example. The auditory vibration sensor 99 includes a condenser microphone, for example.

A signal indicating the tactile vibration detected by the tactile vibration sensor 97 is input into the first vibration transmitting section 70. The first vibration transmitting section 70 generates a driving signal for a vibration generating source 67 of the master apparatus 60 on the basis of the input signal indicating the tactile vibration. Specifically, after the amplifier 71 performs amplification processing on the input signal indicating the tactile vibration, the frequency characteristic correction circuit 73 performs processing for correcting the vibration frequency. In addition, the band-pass filter 75 performs filtering processing. Then, the driving circuit 77 drives the vibration generating source 67 of the master apparatus 60 on the basis of the input signal. Accordingly, the vibration generating source 67 on the master apparatus 60 side generates the vibration corresponding to the tactile vibration detected by the slave apparatus 90, transmitting the tactile vibration generated in the surgical tool to the user. For example, the vibration generating source 67 may include any one or a combination of two or more of a piezo-type vibration actuator, a voice-coil-motor-type vibration actuator, a linear vibration actuator, an ERM (Eccentric Rotating Mass) type vibration actuator, and an EPAM (Electroactive Polymer Artificial Muscle) type vibration actuator.

A signal indicating the auditory vibration detected by the auditory vibration sensor 99 is input into the second vibration transmitting section 80. The second vibration transmitting section 80 outputs a driving signal for a speaker 69 of the master apparatus 60 on the basis of the input signal indicating the auditory vibration. Specifically, after the amplifier 81 performs amplification processing on the input signal indicating the auditory vibration, the frequency characteristic correction circuit 83 performs processing for correcting the vibration frequency. In addition, the band-pass filter 85 performs filtering processing. Then, the driving circuit 87 drives the speaker 69 of the master apparatus 60 on the basis of the input signal. Accordingly, the speaker 69 on the master apparatus 60 side outputs the sound corresponding to the auditory vibration detected by the slave apparatus 90, transmitting the auditory vibration generated in the surgical tool to the user.

It is to be noted that although the master-slave type robot system 1 applied to endoscopic surgery or the like may include constituent elements other than those illustrated in FIG. 1, illustration of these constituent elements is omitted for ease of description.

The master apparatus 60 is the arm apparatus that includes the operation section to be operated by the user and the arm with multiple degrees of freedom. The operation section is attached to the distal end of the arm. By displacing the position and attitude of the operation section, the user can remotely control the positions and attitudes of the arm on the slave apparatus 90 side and the surgical tool such as forceps attached to the end effector of the arm. Further, the master apparatus 60 can present a force sense to the user via the operation section by driving the arm apparatus and displacing the position and attitude of the operation section.

For example, the operation section is connected to the main body of the master apparatus 60 by the arm having a three-axis translational structure, and is rotatably attached to the distal end of the arm. Therefore, the user can move the operation section relative to the main body of the master apparatus 60 while rotationally operating the operation section relative to the distal end of the arm. In this way, the user can give an instruction on the position and attitude of the end effector at the distal end of the arm on the slave apparatus 90 side. In addition, the operation section includes a gripping mechanism. Accordingly, the user can perform the gripping operation on the gripping mechanism, giving an instruction for an opening/closing operation and the like on the forceps attached to the distal end of the arm on the slave apparatus 90 side. Further, the motor 65 is caused to drive the arm or the gripping mechanism of the operation section. Accordingly, the force acting on the slave apparatus 90 side can be presented to the user.

In short, the operation section and the arm on the master apparatus 60 side are input UIs in the robot system 1. Although illustration of the structures of the operation section and the arm on the master apparatus 60 side is omitted in FIG. 1, it is strongly demanded to provide a wide range of motion of the operation section as the input UI since the human arm has a very wide range of motion.

For example, a gimbal structure is widely used as three-axis orthogonal joints. Applying the gimbal structure can configure the operation section capable of performing a rotational operation with three degrees of freedom. However, in a case where the three-axis orthogonal joints are assembled by rotational joints for respective axes, there is an issue that a singularity always occurs in a case where an intermediate axis is rotated by ±90 degrees.

The singularity can be avoided with an operation section having a rotational structure with four degrees of freedom. However, this raises a new issue that the feeling of operation is heavy since the self-weight of a link structure for achieving four degrees of freedom is heavier.

Further, a ball joint structure can be exemplified as another joint structure with three degrees of freedom. As a common structure, however, one half or more of an internal ball is covered in order to prevent the ball from falling, which limits the range of motion to approximately ±30 degrees.

Further, the feeling of operation is light with a parallel link structure. However, a spherical rotational joint using the parallel link structure can only rotate up to ±90 degrees, and it is difficult to further expand the range of motion.

Accordingly, hereinafter, the present specification proposes a lightweight information input apparatus that has a wide range of rotational motion and can input or measure rotation angles of three degrees of freedom. The information input apparatus can be applied as the operation section of the master apparatus 60.

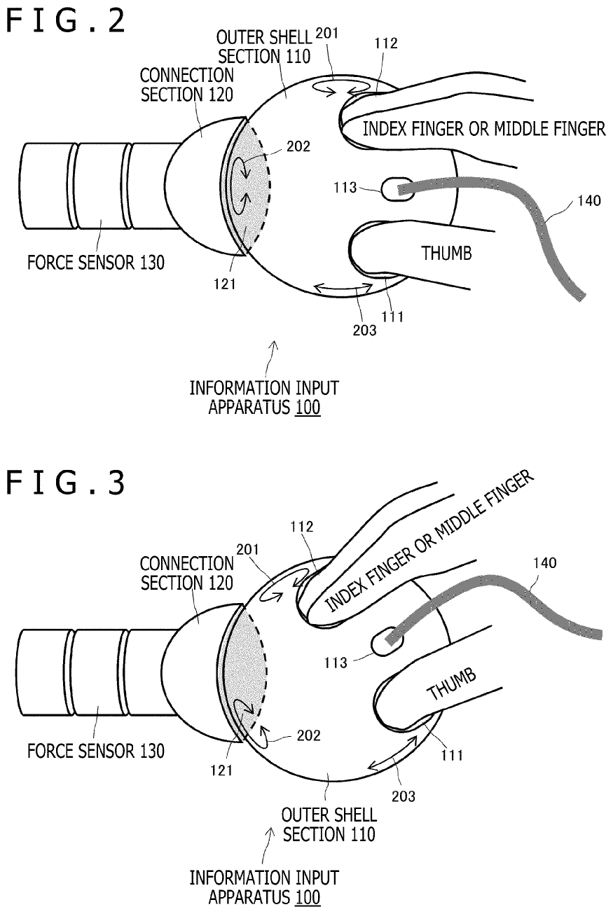

FIG. 2 illustrates an external configuration of an information input apparatus 100 capable of inputting rotation angles of three degrees of freedom proposed in the present specification. The information input apparatus 100 includes an outer shell section 110 and a connection section 120. The outer shell section 110 has a hollow sphere structure. The connection section 120 attracts and rotatably supports a surface of the outer shell section 110. That is, the information input apparatus 100 constitutes a ball joint capable of rotating in three degrees of freedom, as indicated by arrows with reference numerals 201 to 203 in the figure. It is to be noted that the outer shell section 110 may have a sphere structure with only one spherical face or with a plurality of faces coupled to each other.

Basically, the outer shell section 110 includes a magnetic material, while the connection section 120 includes a magnet. The connection section 120 can attract the surface of the outer shell section 110 by the magnetic force of the magnet. The outer shell section 110 can be rotationally operated by sliding relative to the connection section 120 while receiving an attractive force from the magnetic force. It is to be noted that, however, the connection section 120 may attract the outer shell section 110 by using attraction by the air pressure or electrostatic force, instead of the magnetic force of the magnet. In a case where attraction by the air pressure or electrostatic force is used, the outer shell section 110 includes a material with a small specific gravity other than a magnetic material (metal). This allows a reduction in weight.

Friction between the outer shell section 110 and a contact surface 121 of the connection section 120 can be adjusted by devising the roughness and material of the surface. Appropriate friction makes it easy to hold the rotational position of the outer shell section 110 and improves the operability. Further, in order to suppress the torque that is likely to freely rotate due to the self-weight of the outer shell section 110, it is preferable to have appropriate friction between the outer shell section 110 and the contact surface 121 of the connection section 120. It is to be noted that, however, it is preferable to take into account the balance of the center of gravity such that the center of gravity of the outer shell section 110 is positioned in the vicinity of the center of the sphere so as not to generate a rotation moment caused by the self-weight of the outer shell section 110 in the first place.

For example, the contact surface 121 on the connection section 120 side, which is in contact with the outer shell section 110, is subjected to surface finishing or a treatment such as coating to reduce friction. This allows the outer shell section 110 having the sphere structure to easily slide on the contact surface 121 between the outer shell section 110 and the connection section 120, making it easier for the user to rotationally operate the outer shell section 110. Further, instead of the connection section 120 side, the surface of the outer shell section 110 (at least a range that is in contact with the contact surface 121 of the connection section 120) may be subjected to the treatment such as coating to reduce friction. Needless to say, both the contact surface 121 on the connection section 120 side and the surface of the outer shell section 110 may be subjected to low friction coating.

Even if the contact surface between the outer shell section 110 and the connection section 120 is subjected to low friction coating, a frictional sound may be generated and grate on the ears of the user and the surrounding people in some cases. Accordingly, an intermediate member (not illustrated) for generating a frictional sound may be arranged between the outer shell section 110 and the connection section 120. The intermediate member includes a material such as resin, for example. The intermediate member may be fixed to the magnet.

The magnetic force of the magnet is adjusted depending on by how much attractive force the connection section 120 should attract (or pull) the outer shell section 110. Further, the attractive force can be adjusted not only by the magnetic force of the magnet but also by the thickness of coating applied to the contact surface 121 of the connection section 120 or the surface of the outer shell section 110 or by the material arranged between the outer shell section 110 and the connection section 120. Increasing the attractive force can prevent the outer shell section 110 from falling. However, with the outer shell section 110 firmly held by the connection section 120, the outer shell section 110 is difficult to rotate, resulting in a decrease in the operability. Further, decreasing the attractive force can improve the operability of the outer shell section 110, but the outer shell section 110 is more likely to fall from the connection section 120.

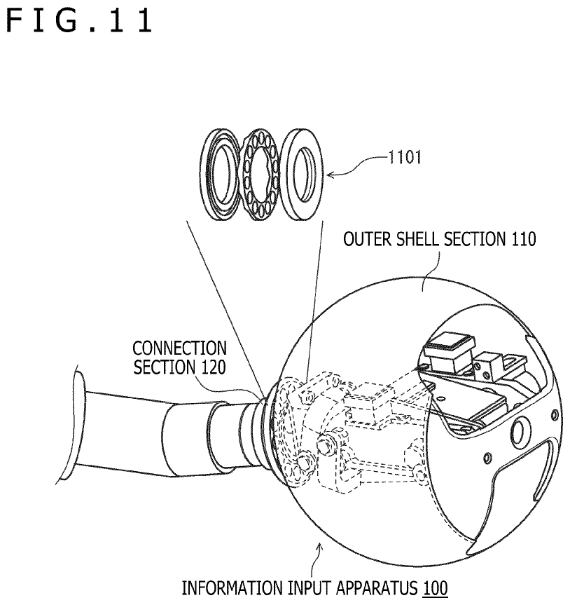

It is to be noted that the outer shell section 110 can also smoothly rotate about the three axes (201 to 203) by using an intermediate member such as a bearing, like a thrust ball bearing 1101, capable of receiving the force acting in the axial direction of the rotational body, as illustrated in FIG. 11, instead of applying low friction coating to the contact surface 121 of the connection section 120 (strictly speaking, of the thrust ball bearing 1101, only a middle holding part that holds balls is necessary, and a shaft washer and a housing washer on both sides are not necessary).

The outer shell section 110 is a sphere having a diameter of approximately 80 mm, for example. As long as the outer diameter of the connection section 120 is equal to or smaller than three-quarters of the diameter of the outer shell section 110, the outer shell section 110 can provide good rotational operability while an appropriate attractive force is maintained. It is to be noted that the diameter of 80 mm is a dimension that assumes that the user is an adult. In a case where a child is a target user, the dimension may be smaller.

The outer shell section 110 includes a first opening section 111 and a second opening section 112 bored on the opposite side of the contact surface 121 between the connection section 120 and the outer shell section 110. The first opening section 111 allows the user's thumb to be inserted therein. The second opening section 112 allows one of or both the user's index finger and middle finger to be inserted therein. Further, the gripping mechanism (not illustrated in FIG. 2) is housed inside the outer shell section 110. This gripping mechanism has an opening/closing structure that allows the user to suitably perform a pinch or gripping operation with the thumb and the index finger or the middle finger. The details will be described later.

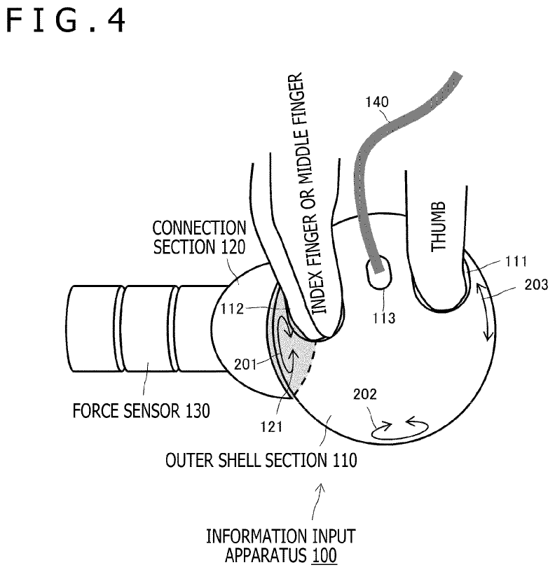

The user can rotationally operate the outer shell section 110 with three degrees of freedom indicated by the arrows with the reference numerals 201 to 203 while performing the pinch operation, that is, gripping the gripping mechanism with the thumb and the index finger or the middle finger inserted through the first opening section 111 and the second opening section, respectively, so as not to release the attraction state by the connection section 120. FIGS. 3 and 4 each illustrate, as an example, a state of the information input apparatus 100 with the outer shell section 110 rotationally operated.

Although illustration is omitted in FIGS. 2 to 4, a rotation angle sensor (corresponding to the rotation angle sensor 63 in FIG. 1), an actuator (corresponding to the motor 65 in FIG. 1), an encoder (corresponding to the rotation angle sensor 63 in FIG. 1), tactile presentation actuators (each corresponding to the vibration generating source 67 in FIG. 1), finger detection sensors, and the like are housed inside the outer shell section 110. The rotation angle sensor detects the three-axis rotation angles of the outer shell section 110. The actuator causes the gripping mechanism to perform the opening/closing operation. The encoder detects the rotation angle of the opening/closing structure. The tactile presentation actuators present a tactile sense on surfaces of the gripping mechanism that are in contact with the user's thumb and index finger or middle finger. The finger detection sensors detect that the user's thumb and index finger or middle finger have been inserted into the outer shell section 110.

When the user rotationally operates the outer shell section 110 while gripping the gripping mechanism with the thumb and the index finger or the middle finger so as not to release the attraction state by the connection section 120, the above-described rotation angle sensor detects the three-axis rotation angles of the outer shell section 110. The three-axis rotation angles detected by the rotation angle sensor serve as information indicating an instruction to drive the arm and the end effector of the arm on the slave apparatus 90 side.

Further, the encoder can detect the rotation angle of the opening/closing structure when the user performs the pinch operation with the thumb and the index finger or the middle finger, that is, when the user grips the gripping mechanism. For example, in a case where the medical surgical tool such as forceps is attached to the end effector of the arm on the slave apparatus 90 side, the rotation angle detected by the encoder serves as information indicating an instruction to drive the surgical tool.

Further, the actuator of the gripping mechanism is driven to cause the gripping mechanism to perform the opening/closing operation. Accordingly, the gripping force can be presented to the user's thumb and index finger or middle finger gripping the gripping mechanism. For example, when the medical surgical tool such as forceps is attached to the end effector of the arm on the slave apparatus 90 side and the user gives an instruction to drive the surgical tool by gripping the gripping mechanism with the thumb and index finger or middle finger as described above and performing the opening/closing operation, the slave apparatus 90 side extracts a component of the force acting on the surgical tool from the forces acting on the respective joint portions detected by the force sensors 91 and calculates the control amount of the motor of the gripping mechanism. Then, the motor is driven according to the calculated control amount. Accordingly, the force acting on the surgical tool can be presented to the user.

Further, the tactile vibration sensor 97 on the slave apparatus 90 side detects the tactile vibration generated in the surgical tool. After this tactile signal is subjected to signal processing (described above) by the first vibration transmitting section 70, the tactile signal is input into the tactile presentation actuators in the outer shell section 110 as a driving signal. Therefore, the tactile presentation actuators in the outer shell section 110 can generate vibration corresponding to the tactile vibration detected by the slave apparatus 90 (for example, the end effector), transmitting the tactile vibration generated in the surgical tool to the user.

Further, the finger detection sensors detect that the user's thumb and index finger or middle finger have been inserted into the outer shell section 110. Whether or not the information input apparatus 100 is in use can be determined on the basis of detection signals.

Various circuit parts other than the motors, actuators, and sensors described above can also be housed inside the outer shell section 110 as necessary. Further, a wire hole 113 into which a wire 140 is inserted is bored in the outer shell section 110. The wire 140 is used to electrically connect electronic parts housed in the outer shell section 110 to the outside and includes signal lines for supplying control signals to the motor for the gripping mechanism and the tactile presentation actuators and signal lines for outputting a detection signal of each sensor to the outside. Preferably, the wire hole 113 is formed in the vicinity of a midpoint between the first opening section 111 and the second opening section 112, and the wire 140 passes between the user's thumb and index finger or middle finger.

The details of an internal configuration and layout of the outer shell section 110 will be described later. It is to be noted that, however, all of the rotation center of the outer shell section 110 having the sphere structure, the center of gravity of the outer shell section 110, and the gripping center gripped by the user with the thumb and the index finger or the middle finger are preferably positioned at approximately the same point.

Arranging the center of gravity of the outer shell section 110 in the vicinity of the center of the sphere can suppress the rotation moment caused by the center of gravity of the outer shell section 110 and prevent such a malfunction and false detection that the outer shell section 110 freely (spontaneously) rotates even though the user does not operate the outer shell section 110. Further, the user does not receive extra torque when rotationally operating the outer shell section 110. This results in improved operability and reduced fatigue.

Further, the gripping center gripped by the user with the thumb and the index finger or the middle finger is arranged in the vicinity of the center of the sphere. Accordingly, when the user performs the pinch operation on the gripping mechanism in the outer shell section 110, it is possible to prevent the influence of a change in the attitudes of the user's fingers from affecting a change in the position of the outer shell section 110.

In a case where the information input apparatus 100 illustrated in FIGS. 2 to 4 is used as the operation section in the master apparatus 60 of the robot system 1, the information input apparatus 100 is coupled to the arm (illustration is omitted) having the three-axis translational structure coupled to the main body of the master apparatus 60 via a force sensor 130. The force sensor 130 detects an external force acting on the operation section.

The force sensor 130 corresponds to the force sensor 61 in FIG. 1 and includes a six-axis force sensor, for example. The force sensor 130 detects an external force acting on the outer shell section 110 when the user performs an operation, for example. Moreover, translation forces Fx, Fy, and Fz in the three-axis XYZ directions orthogonal to each other and moments Mx, My, and Mz about the respective axes can be obtained by appropriately computing a detection signal of the force sensor 61. A force that the user acts on the outer shell section 110 may be detected using the force sensor 130 and used to control the arm and the end effector on the slave apparatus 90 side. At this time, a force by the friction between the outer shell section 110 and the connection section 120 and a generative force by the user's fingers may be separated. It is to be noted that, however, even in a case where a three-axis force sensor is used as the force sensor 130 instead of the six-axis force sensor, the equivalent effect can be obtained.

It should be fully understood that the information input apparatus 100 is an input device that allows the user to intuitively perform an input operation and has a wide range of motion with reference to FIGS. 2 to 4. Further, the user can perform the input operation by inserting at least two fingers including the thumb into the outer shell section 110. Thus, it can also be said that the information input apparatus 100 is an easy-to-use input device for humans.

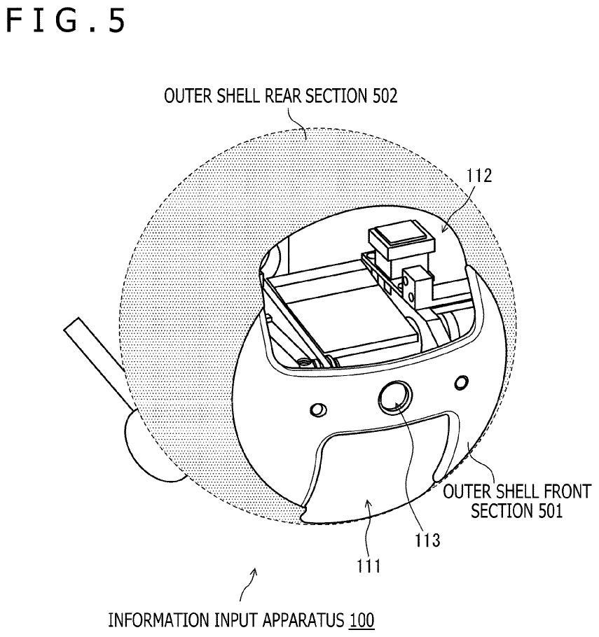

FIG. 5 illustrates a specific example of the configuration of the information input apparatus 100. It is to be noted that, however, this figure illustrates an obliquely viewed external appearance of a portion in which the first opening section 111 and the second opening section 112 are bored in the outer shell section 110.

Since the outer shell section 110 has the internal structure (housing various parts including the gripping mechanism) as described above, it is difficult to manufacture the sphere structure with a single-piece part. Therefore, a realistic manufacturing method is to divide the sphere into two or more portions, insert the internal structure from part of the portions, and then couple these portions to each other to assemble the sphere structure. Meanwhile, the outer shell section 110 needs to rotate smoothly while the outer shell section 110 is kept attracted to the contact surface 121 of the connection section 120 using the magnetic force of the magnet or the like. For this reason, it is strongly desired that the outer shell section 110 is seamless, that is, a single-piece part in a range (range of motion) in which the outer shell section 110 may slide on the contact surface 121 of the connection section 120.

In the present embodiment, therefore, the outer shell section 110 has a structure including two divided bodies of an outer shell front section 501 and an outer shell rear section 502. With the outer shell front section 501 and the outer shell rear section 502 disassembled (or not joined together), the internal structure can be inserted. While the outer shell rear section 502 includes a region that slides on the contact surface 121 of the connection section 120, the outer shell section 110 does not slide on the contact surface 121 of the connection section 120 in the outer shell front section 501. The outer shell rear section 502 is illustrated with a dot pattern in FIG. 5. Since the outer shell rear section 502 is a seamless single-piece part, the outer shell rear section 502 can smoothly move on the contact surface 121 of the connection section 120. Since the outer shell front section 501 is not attracted by the magnetic force, the outer shell front section 501 may include a material other than metal to reduce the weight.

The outer shell section 110 can be assembled by coupling the outer shell front section 501 to the outer shell rear section 502 after attaching the internal structure inside the outer shell section 110 with the outer shell front section 501 detached. Alternatively, the outer shell section 110 can be assembled by attaching the outer shell front section 501 to the outer shell rear section 502 after assembling the internal structure to (an inner wall of) the outer shell front section 501.

The first opening section 111 and the second opening section 112 are formed using a boundary between the outer shell front section 501 and the outer shell rear section 502. The second opening section 112 may be formed larger than the first opening section 111. Further, the wire hole 113 is bored in the vicinity of the center between the first opening section 111 and the second opening section 112 in the outer shell front section 501. The wire (not illustrated in FIG. 5) including the signal lines that electrically connect the electronic parts housed in the outer shell section 110 to the outside is inserted into the wire hole 113. For example, after the wire extending from the internal structure is inserted into the wire hole 113, the internal structure is assembled to (the inner wall of) the outer shell front section 501. Then, the outer shell front section 501 is attached to the outer shell rear section 502. In this way, the outer shell section 110 can be assembled.

As described above, the gripping mechanism, the motor, the encoder, the rotation angle sensor, the tactile presentation actuators, the finger detection sensors, and the like are housed inside the outer shell section 110. The gripping mechanism can be gripped by the user with the thumb and the index finger or the middle finger. The motor causes the gripping mechanism to perform the opening/closing operation. The encoder detects the rotation angle of the gripping mechanism. The rotation angle sensor detects the three-axis rotation angles of the outer shell section 110. The tactile presentation actuators present a tactile sense to the user's thumb and index finger or middle finger gripping the gripping mechanism. The finger detection sensors detect that the user's thumb and index finger or middle finger have been inserted into the outer shell section 110.

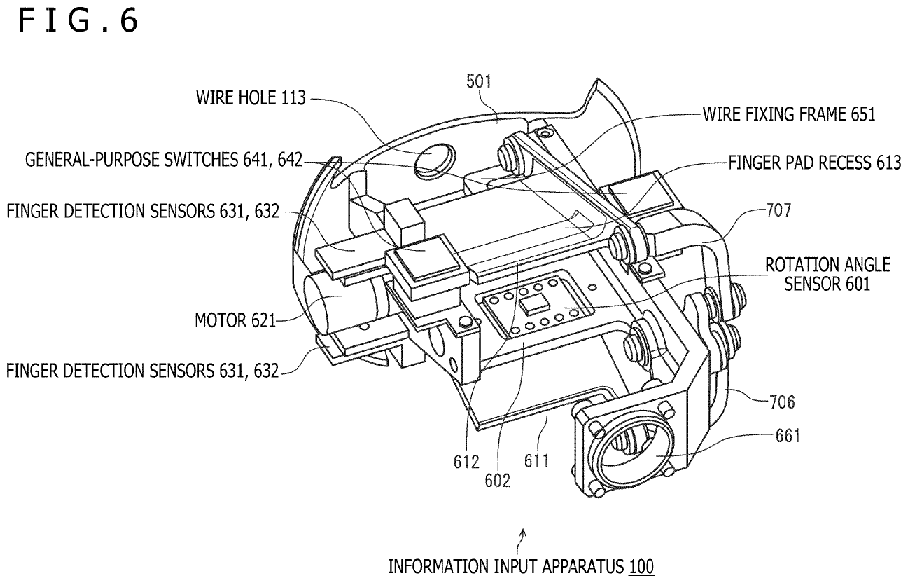

FIG. 6 illustrates a specific example of an internal configuration of the information input apparatus 100. It is to be noted that, however, this figure illustrates a state in which the inside of the information input apparatus 100 with the outer shell rear section 502 detached is obliquely viewed from the back side of the information input apparatus 100, that is, the side connected to the connection section 120.

A rotation angle sensor 601, the gripping mechanism, a motor 621, the encoder, the tactile presentation actuators, finger detection sensors 631 and 632, and the like are included inside the outer shell section 110. The rotation angle sensor 601 detects the three-axis rotation angles of the outer shell section 110. The gripping mechanism can be gripped by the user with the thumb and the index finger or the middle finger. The motor 621 causes the gripping mechanism to perform the opening/closing operation. The encoder detects the rotation angle of the gripping mechanism. The tactile presentation actuators present a tactile sense to the user's thumb and index finger or middle finger gripping the gripping mechanism. The finger detection sensors 631 and 632 detect that the user's thumb and index finger or middle finger have been inserted into the outer shell section 110.

The rotation angle sensor 601 is mounted on a surface of a substrate section 602 fixed to the outer shell front section 501. The rotation angle sensor 601 includes an IMU (Inertial Measurement Unit), and is arranged approximately at the center of the sphere constituting the outer shell section 110. The rotation angle sensor 601 can detect three-dimensional acceleration and angular velocity that act on the outer shell section 110 (or the main body of the information input apparatus 100).

The IMU basically includes a three-axis gyro sensor, a three-axis geomagnetic sensor, and a three-directional acceleration sensor. Incidentally, the high-speed operation of the outer shell section 110 for a short time can be measured using the gyro sensor. On the other hand, the drift that occurs for a long time can be measured by using both the acceleration sensor and the geomagnetic sensor. That is, drift in a horizontal direction can be corrected by measuring both the acceleration sensor and the geomagnetic sensor. Further, the rotational drift about a gravity-direction axis can be corrected by measuring a magnetic field generated by the magnet of the connection section 120 for attracting the outer shell section 110.

It is to be noted that arranging the IMU in the vicinity of the center of the sphere of the outer shell section 110 can suppress the influence on the acceleration sensor when the outer shell section 110 is rotationally operated relative to the connection section 120. Further, the magnet attracting the outer shell section 110 is fixed in one direction. With this structure, arranging the IMU in the vicinity of the center of the sphere allows the geomagnetic sensor to estimate the current angle.

Alternatively, the rotation angle sensor 601 can include a camera (not illustrated) instead of the IMU. The camera is installed on the connection section 120 side or outside the apparatus. The camera images a pattern formed on an outer wall of the outer shell section 110 or a direction of the operator's hand. Then, these subjects are tracked by image analysis, through which the rotation angle of the outer shell section 110 can be detected.

Further, the substrate section 602 may include a circuit part (not illustrated) other than the rotation angle sensor 601 such as the IMU, or may have a surface on which a wiring pattern (not illustrated) is formed.

The gripping mechanism includes a first gripping plate 611 and a second gripping plate 612. The user's thumb inserted from the first opening section 111 abuts against the first gripping plate 611. One of or both the user's thumb and index finger or middle finger inserted from the second opening section 112 abut against the second gripping plate 612. A front end portion of each of the first gripping plate 611 and the second gripping plate 612 is turnably axially supported by the outer shell front section 501 or the substrate section 602. Therefore, the opening/closing operation is realized by rotating the first gripping plate 611 and the second gripping plate 612 in the mutually opposite directions about the respective front-end turning axes.

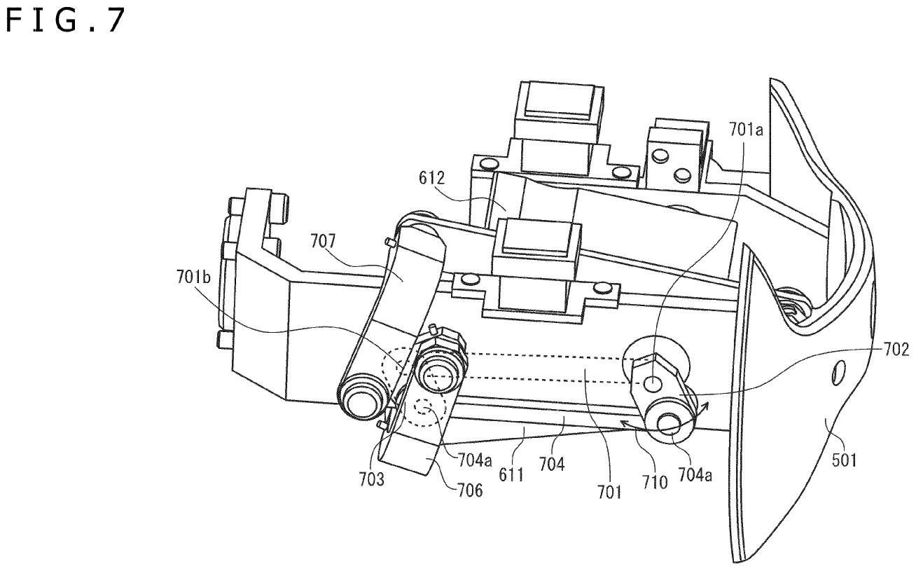

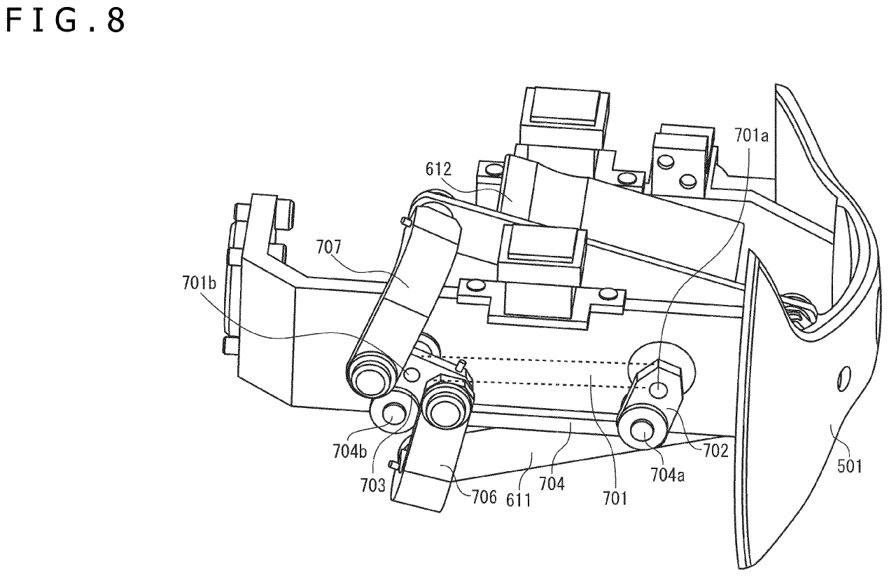

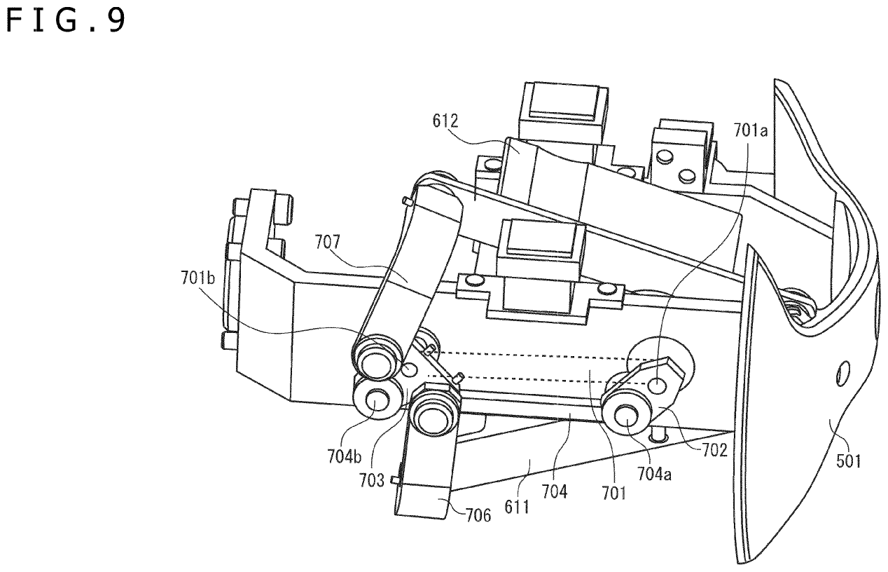

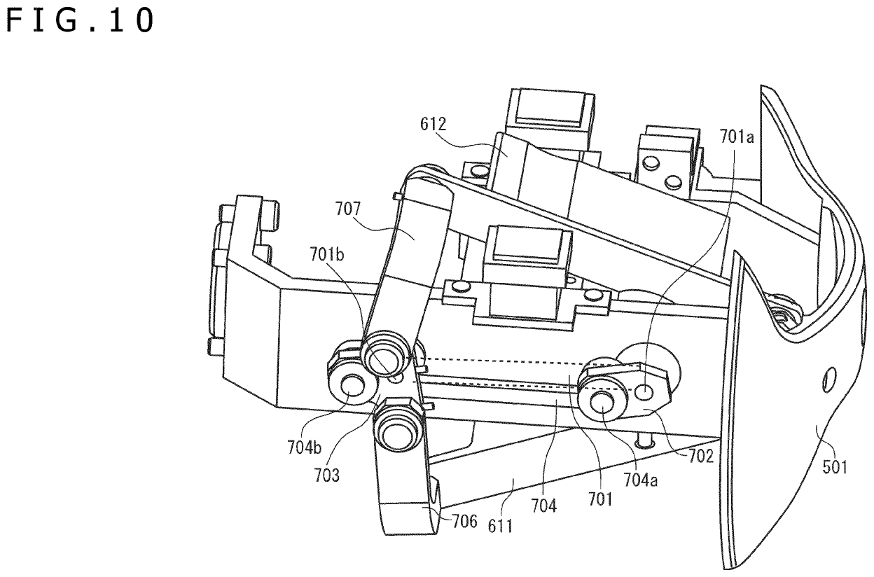

In the present embodiment, the rotational movement of a four-link mechanism is used to realize the gripping operation of the gripping mechanism, that is, the opening/closing operation of the first gripping plate 611 and the second gripping plate 612. Here, the gripping operation of the gripping mechanism using the rotational movement of the four-link mechanism will be described with reference to FIGS. 7 to 10. However, it should be fully understood that a configuration other than the four-link mechanism can also be used to realize the gripping mechanism.

The four-link mechanism referred to herein is a fixed link 701 and includes a driving link 702, a driven link 703, and an intermediate link 704. The fixed link 701 includes part of the substrate section 602 on which the IMU or the like is mounted. The driving link 702 is turnably coupled to one joint shaft (driving shaft) 701a and receives a driving force from the motor 621 (described above; not illustrated in FIGS. 7 to 10). The joint shaft (driving shaft) 701a is fixed to one end of the fixed link 701. The driven link 703 is turnably coupled to a joint shaft (driven shaft) 701b and faces the driving link 702. The joint shaft (driven shaft) 701b is fixed to the other end of the fixed link 701. The intermediate link 704 turnably connects the driving link 702 and the driven link 703 using joint shafts 704a and 704b, respectively.

When the driving link 702 receives the driving force from the motor 621, the driving link 702 turns about the driving shaft 701a as indicated by an arrow with reference numeral 710 to swing the intermediate link 704. Further, it is assumed that the motor 621 includes the encoder (not illustrated) for detecting the rotation angle of an output shaft.

Meanwhile, the driven link 703 has a T-shape with branch portions in which links orthogonal to the driven link 703 extend from the respective right and left sides of the driven shaft 701b. A first transmission link 706 and a second transmission link 707 are turnably axially supported at both ends of the respective branch portions. The first transmission link 706 and the second transmission link 707 are coupled to the respective rear ends of the first gripping plate 611 and the second gripping plate 612, respectively.

When the driving link 702 receives the driving force from the motor 621 in a rotation direction indicated by the arrow 710, the driven link 703 is driven via the intermediate link 704. Then, when the T-shaped driven link 703 turns about the driven shaft 701b, the rear ends of the first gripping plate 611 and the second gripping plate 612 are pulled toward each other (see FIG. 7) or conversely pulled away from each other (see FIG. 10) by the first transmission link 706 and the second transmission link 707 according to the rotation angle of the T shape. In this way, the opening/closing operation of the gripping mechanism is realized.

With reference to FIGS. 7 to 10, when the driving link 702 is driven by the motor 621 clockwise on the paper, the gripping mechanism, that is, the first gripping plate 611 and the second gripping plate 612 open. Conversely, when the driving link 702 is driven by the motor 621 counterclockwise on the paper, the gripping mechanism closes. Further, the encoder included in the motor 621 successively detects the rotation angle of the gripping mechanism.

The motor 621 is driven to cause the gripping mechanism to perform the opening/closing operation. Accordingly, the gripping force can be presented to the user's thumb and index finger or middle finger holding the first gripping plate 611 and the second gripping plate 612. Further, the encoder detects the rotation angle of the gripping mechanism when the user performs the pinch operation with the thumb and the index finger or the middle finger. The rotation angle detected by the encoder serves as information indicating an instruction to drive the end effector (for example, the medical surgical tool such as forceps) of the arm on the slave apparatus 90 side.

It is to be noted that the output shaft of the motor 621 does not need to be directly coupled to the driving shaft 701a of the above-described four-link mechanism and can be arranged away from the driving shaft 701a using a transmission mechanism (not illustrated). The ratio of the weight of the motor 621 to the weight of the entire outer shell section 110 is high and the arrangement of the motor 621 greatly affects the center of gravity of the outer shell section 110. It is preferable to take into account the balance of the center of gravity such that the center of gravity of the outer shell section 110 is positioned in the vicinity of the center of the sphere so as not to generate a rotation moment caused by the self-weight of the outer shell section 110 (as described above). A more preferable design is, therefore, such that the center of gravity of the outer shell section 110 including the motor 621 is positioned in the vicinity of the center of the sphere. For example, arranging the motor 621 closer to the outer shell front section 501 (or in the vicinity of the opening section 111 or 112) facilitates maintaining the weight balance with the outer shell section 110.

The internal configuration of the information input apparatus 100 is continuously described with reference to FIG. 6 again.

A finger pad recess 613 is formed on the surface of each of the first gripping plate 611 and the second gripping plate 612 that is in contact with the user's index finger or middle finger. Further, although not visible in FIG. 6, the first gripping plate 611 side also has the finger pad recess formed on the surface thereof that is in contact with the user's thumb. When the user inserts the user's thumb and index finger or middle finger from the opening sections 111 and 112, respectively, the user cannot see the inside. However, the user can find suitable locations for the gripping operation on the respective gripping plates by searching for the finger pad recesses 613 with fingertips.

The gripping mechanism is arranged such that when the user performs the pinch operation on the gripping mechanism with the thumb and the index finger or the middle finger, the center between the fingers (the thumb and the index finger or the middle finger) is positioned in the vicinity of the center position of the sphere constituting the outer shell section 110. With this arrangement, it is possible to prevent the influence of a change in the attitudes of the user's fingers upon gripping from affecting a change in the position of the outer shell section 110. Further, with the finger pad recesses 613 formed in appropriate locations on the first gripping plate 611 and the second gripping plate 612, the user aligns the center between the user's thumb and index finger or middle finger with the locations of the finger pad recesses 613 and performs the gripping operation. Accordingly, it is possible to prevent the influence of a change in the attitudes of the user's fingers upon gripping from affecting a change in the position of the outer shell section 110.

Further, although illustration is omitted in FIG. 6, the tactile presentation actuators that present a tactile sense are arranged on the respective surfaces of the first gripping plate 611 and the second gripping plate 612 that are in contact with the user's thumb and index finger or middle finger. Each tactile presentation actuator corresponds to the vibration generating source 67 in FIG. 1. For example, each tactile presentation actuator includes any one or a combination of two or more of a piezo-type vibration actuator, a voice-coil-motor-type vibration actuator, a linear vibration actuator, an ERM-type vibration actuator, and an EPAM-type vibration actuator. With the tactile presentation actuators arranged in the locations through which the finger pad recesses 613 pass, the tactile sense can be surely presented to the user's fingertips.

The finger detection sensor 631 is arranged on a side edge of the first gripping plate 611 and detects that the user's finger (thumb) inserted from the first opening section 111 has been placed on the first gripping slab 611. Similarly, the finger detection sensor 632 is arranged on a side edge of the second gripping plate 612 and detects that the user's finger (index finger or middle finger) inserted from the second opening section 112 has been placed on the second gripping slab 612. The finger detection sensors 631 and 632 can include optical sensors such as photo reflectors, capacitance sensors, or other human sensors, for example. Whether or not the information input apparatus 100 is in use can be determined on the basis of detection signals of the finger detection sensors 631 and 632.

General-purpose switches 641 and 642 include switches of a seesaw type, a push type, a slide type, or the like that can be operated by the user with a fingertip. The user can operate the general-purpose switches 641 and 642 with the index finger or the middle finger inserted from the second opening section 112. Applications of the general-purpose switches 641 and 642 are arbitrary. The general-purpose switches 641 and 642 can be used to input instructions other than the three-axis rotation angles.

The wire hole 113 (described above) is bored approximately in the center of the outer shell front section 501. The wire (illustration is omitted in FIG. 6) for electrically connecting the electronic parts housed in the outer shell section 110 to the outside is inserted into the wire hole 113. Further, a wire fixing frame 651 fixes the wire to prevent the wire from interfering with the operation of the gripping mechanism with the user's fingers.

A spherical assembling section 661 is arranged at a tail end of the substrate section 602 (on the front side of the paper). The outer shell rear section 502 is attached to the spherical assembling section 661. Illustration of the outer shell rear section 502 is omitted in FIG. 6.

A battery (not illustrated) that supplies power to the internal parts of the outer shell section 110 may be further housed inside the outer shell section 110. Alternatively, power may be wirelessly fed to the internal parts of the outer shell section 110, and a wireless communication section for performing wireless power feeding may be further included as an internal part. The battery is a heavy object. Therefore, in a case where the battery is housed inside the outer shell section 110, it is preferable to determine the arrangement location of the battery while taking into account the balance of the center of gravity such that the center of gravity of the outer shell section 110 having the sphere structure does not deviate from the vicinity of the center of the sphere.

The information input apparatus 100 according to the present embodiment can detect rotations about the three axes, detect the user's gripping force, and present the gripping force. The information input apparatus 100 has the configuration in which the outer shell section 110 having the sphere structure, which is to be operated by the user, is attracted and connected by the magnetic force of the magnet or the like. The information input apparatus 100 has no singularity like the gimbal structure and can be used as a rotation input UI with three degrees of freedom that has a wide range of motion. Basically, the entire surface of the outer shell rear section 502 serves as a range of motion and has no singularity.

In the case of employing the configuration in which the outer shell section 110 (outer shell rear section 502) including the magnetic material is attracted and connected by the magnet, a reaction force acts when the magnet has reached a boundary portion between the outer shell rear section 502 and the outer shell front section 501. Thus, the limit of the range of motion can be softly presented to the user operating the outer shell section 110.

Further, the information input apparatus 100 has the configuration in which the rotation angle sensor including the IMU, the camera, or the like and housed inside the outer shell section 110 measures the three-axis rotation angles when the outer shell section 110 having the sphere structure, which is operated by the user, moves on the contact surface 121 of the connection section 120. Therefore, it is not necessary to mount a bearing or a joint angle sensor for each axis. As a result, the small and lightweight information input apparatus 100 can be designed and manufactured.

Further, the rotation moment caused by the self-weight of the outer shell section 110 can be suppressed by taking into account the balance of the center of gravity such that the center of gravity of the outer shell section 110 having the sphere structure is positioned in the vicinity of the center of the sphere. As a result, it is possible to reduce a risk that when the user removes the user's hand from the outer shell section 110, the outer shell section 110 rotates by its self-weight, resulting in an unintentional attitude change. Further, the user does not receive extra torque when rotationally operating the outer shell section 110. This results in improved operability and reduced fatigue.

In a case where the rotation angle sensor of the outer shell section 110 includes the IMU, the IMU is arranged in the vicinity of the center of the sphere of the outer shell section 110. This arrangement can suppress the influence on the acceleration sensor when the outer shell section 110 is rotationally operated. Further, the magnet attracting the outer shell section 110 is fixed in one direction. With this structure, arranging the IMU in the vicinity of the center of the sphere allows the geomagnetic sensor to estimate the current angle.

The information input apparatus 100 has a simple connection structure in which the outer shell section 110 having the sphere structure, which is to be operated by the user, is attracted by the magnetic force of the magnet or the like. Therefore, when an excessive force is applied to the outer shell section 110, the outer shell section 110 overcomes the attractive force of the magnet and is detached from the connection section 120. Therefore, the information input apparatus 100 can be prevented from being broken or damaged. Further, in a use case where the information input apparatus 100 is applied as the operation section at the distal end of the master apparatus 60 (described later) having the three-axis translational structure, even in a case where the master apparatus 60 unintentionally generates an excessive force (for example, in case of runaway), the outer shell section 110 is detached from the connection section 120. Thus, the user can be prevented from being injured during operation.

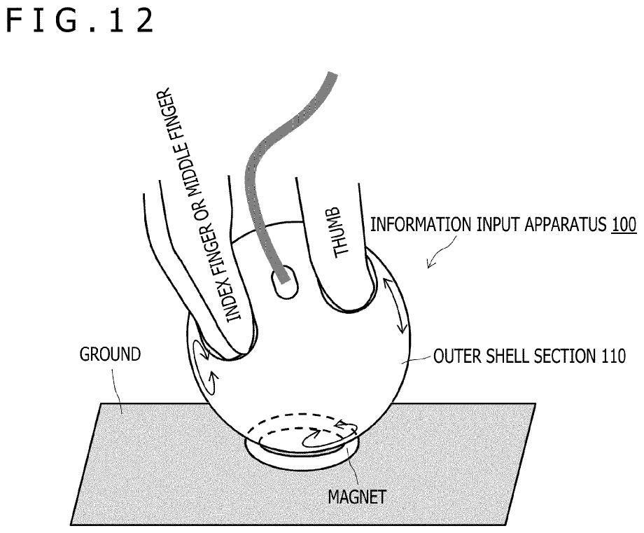

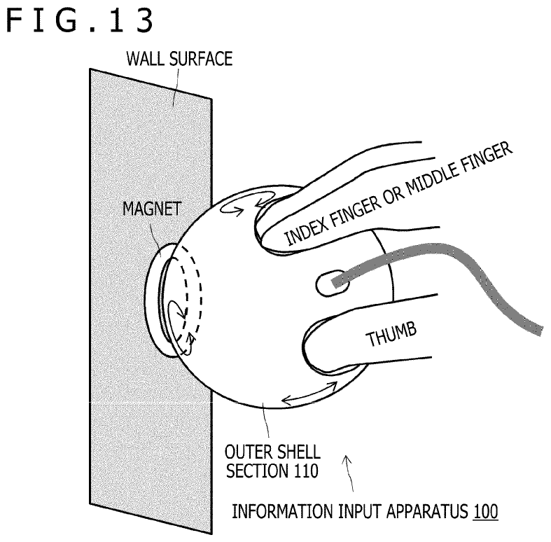

The information input apparatus 100 has a relatively simple structure in which the surface of the outer shell section having the hollow sphere structure is attracted and rotatably supported by the magnetic force of the magnet. Therefore, the outer shell section 110 can be directly attached to the ground or a wall surface by the magnet, and the information input apparatus 100 can be used as a simple UI for inputting only the attitude about the three axes. FIG. 12 illustrates a state in which the outer shell section 110 of the information input apparatus 100 is directly attached to the ground. Further, FIG. 13 illustrates a state in which the outer shell section 110 of the information input apparatus 100 is directly attached to a wall surface.

Further, the information input apparatus 100 can be used as a distal end structure of a six-axis input UI for operating a robot or VR (Virtual Reality) in combination with the three-axis translational structure, for example.

Specifically, the information input apparatus 100 can be used as the operation section on the master apparatus 60 side of the master-slave type robot system 1 (see FIG. 1). For example, the information input apparatus 100 is attached as the operation section at the distal end of the master apparatus 60 having the three-axis translational structure. The main body of the master apparatus 60 can provide functions of detecting translation forces and presenting the translation forces, while the information input apparatus 100 serving as the operation section can provide functions of detecting torque, detecting a gripping force, and presenting the gripping force.

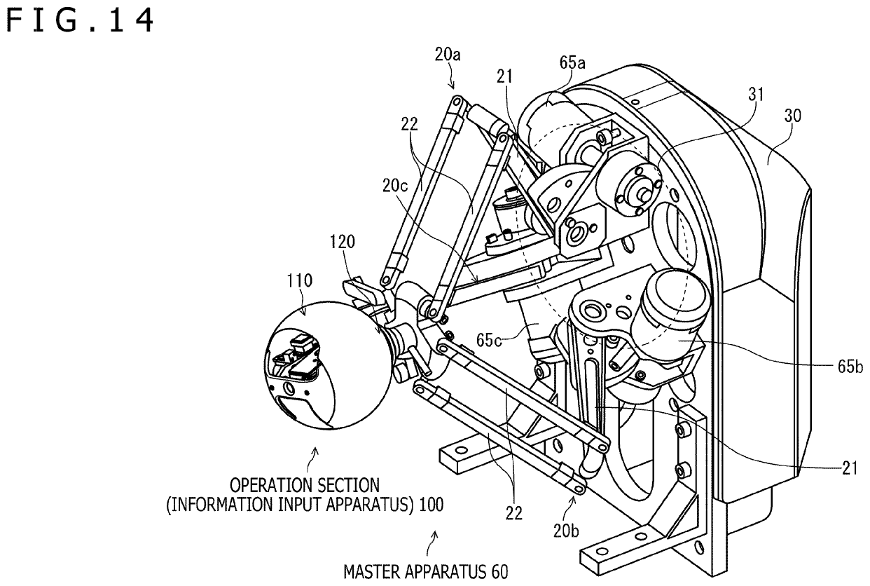

FIG. 14 illustrates a perspective view of the master apparatus 60 of the robot system 1 to which the information input apparatus 100 illustrated in FIGS. 2 to 4 is applied as the operation section. The master apparatus 60 illustrated as an example in FIG. 14 includes a main body section 30, an operation section (information input apparatus) 100, and a support arm section 20. It is to be noted that illustration of the wire that is inserted into the wire hole at the distal end of the operation section 100 is omitted in FIG. 14.

The support arm section 20 has a delta-type parallel link structure including three link sections 20a to 20c and has the three-axis translational structure. Each of the link sections 20a to 20c is turnably coupled to the main body section 30 on the proximal end side. The main body section 30 includes motors (for example, servomotors) 65a to 65c, which each drive a coupling portion with the corresponding one of the link sections 20a to 20c. Further, encoders (illustration is omitted in FIG. 14; corresponding to the rotation angle sensors 63 in FIG. 1) are arranged at the coupling portions between the main body section 30 and the respective link sections 20a to 20c. Each encoder detects the rotation angle of the corresponding one of the link sections 20a to 20c relative to the main body section 30.

Further, the operation section 100 is attached to the distal end side of each of the link sections 20a to 20c. Specifically, the operation section 100 includes the outer shell section 110 having the sphere structure and the connection section 120 rotatably attracting the outer shell section 110 (as described above). The outer shell section 110 is rotatably supported via the connection section 120 on the distal end side of each of the link sections 20a to 20c. The coupling portions between the respective link sections 20a to 20c and the connection section 120 are also turnably coupled. Further, although illustration is omitted in FIG. 14 for simplification, the connection section 120 includes the force sensor 130 described above.

The individual link sections 20a to 20c are separately arranged at intervals of approximately 120 degrees on a circumference with the same radius centered at a center point (illustration is omitted) that is set on an attachment surface 31 of the main body section 30. Therefore, the support arm section 20 is substantially symmetrical with respect to an axis passing through the attachment surface 31 at this center point.

Here, each of the link sections 20a to 20c includes a driving link 21 and a pair of passive links 22. The driving link 21 extends outward in a radial direction radially extending from the above-described center point on the attachment surface 31 of the main body section 30. One end of each driving link 21 is coupled to an output shaft of the corresponding one of the motors 65a to 65c. Moreover, with the axis passing through the above-described center point serving as the center, each link 21 is turnable in a vertical plane orthogonal to the attachment surface 31 and including the axis.

One end of the pair of passive links 22 is turnably coupled to the other end of the corresponding driving link 21. Further, the connection section 120 is attached to the other end (distal end side) of the pair of passive links 22, as described above.

Therefore, synchronously driving each of the motors 65a to 65c turns the driving links 21 and the passive links 22 in the vertical plane. As a result, the operation section 100 coupled to the distal end side of each of the link sections 20a to 20c can be displaced to any position in a three-dimensional space.

The user can displace the operation section 100 to any position in the three-dimensional space by inserting the thumb and the index finger or the middle finger into the outer shell section 110 and performing the pinch operation. Moreover, the rotation angle of each of the link sections 20a to 20c can be detected by the corresponding one of the encoders arranged at the coupling portions between the main body section 30 and the respective link sections 20a to 20c (driving links 21). For example, in a case where the medical surgical tool such as forceps is attached to the end effector of the arm on the slave apparatus 90 side, a signal indicating the rotation angle of the driving link 21 of each of the link sections 20a to 20c is transmitted to the control apparatus 79 as information indicating an instruction to displace the surgical tool.

Further, driving each of the motors 65a to 65c arranged at the coupling portions between the main body section 30 and the respective link sections 20a to 20c can present translation forces to the user pinching the gripping mechanism (described above) in the outer shell section 110. For example, when the surgical tool on the slave apparatus 90 side is displaced, a component of the force acting on the surgical tool is extracted from the forces acting on the respective joint portions detected by the force sensors 91, and the control amount of each of the motors 65a to 65c that drive the coupling portions with the driving links 21 of the respective link sections 20a to 20c is calculated. Then, each of the motors 65a to 65c is driven according to the calculated control amount. Accordingly, the translation force acting on the surgical tool can be presented to the user.

Therefore, in the master apparatus 60 illustrated in FIG. 14, the operation section (information input apparatus) 100 attached to the distal end portion can provide functions of detecting torque, detecting a running force, and presenting a gripping force, while the main body of the master apparatus 60 can provide functions of detecting and presenting the three-axis translation forces.

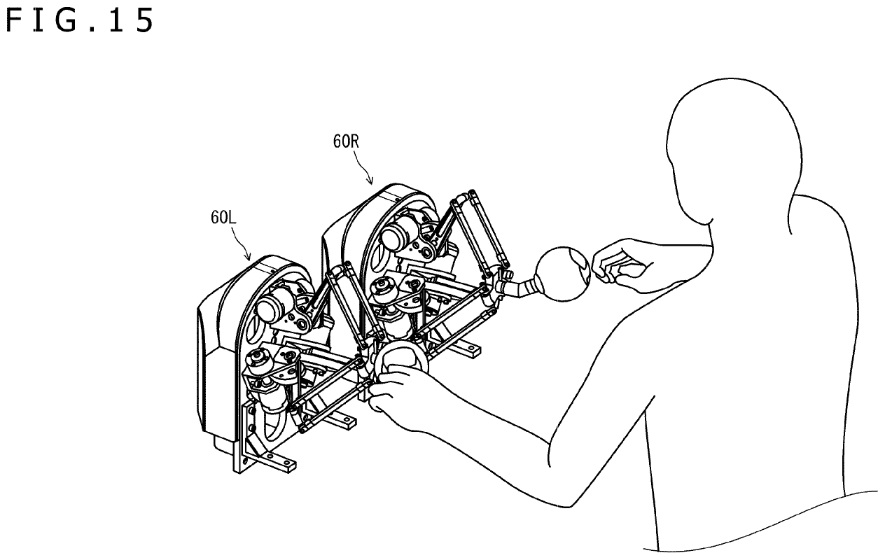

The master apparatus 60 illustrated in FIG. 14 is operated by the user using either the left or right arm and hand. In a case where the master-slave type robot system 1 is applied to endoscopic surgery or the like, a pair of left and right master apparatuses 60L and 60R should be installed as illustrated in FIG. 15 to allow the user as a surgeon to perform surgery using both left and right arms and hands.

Here, it is preferable that in consideration of ergonomics, the operation section (information input apparatus) 100 is attached in each of the master apparatuses 60L and 60R in the attitude that facilitates the user's operation using each of the left and right arms and hands. Attaching the operation section (information input apparatus) 100 at an appropriate angle at each of the distal end portions of the master apparatuses 60L and 60R facilitates the user's operation, allowing the user to work with high accuracy. Further, since the user is less likely to feel fatigue in the arms and hands, the user can endure even long-time work.

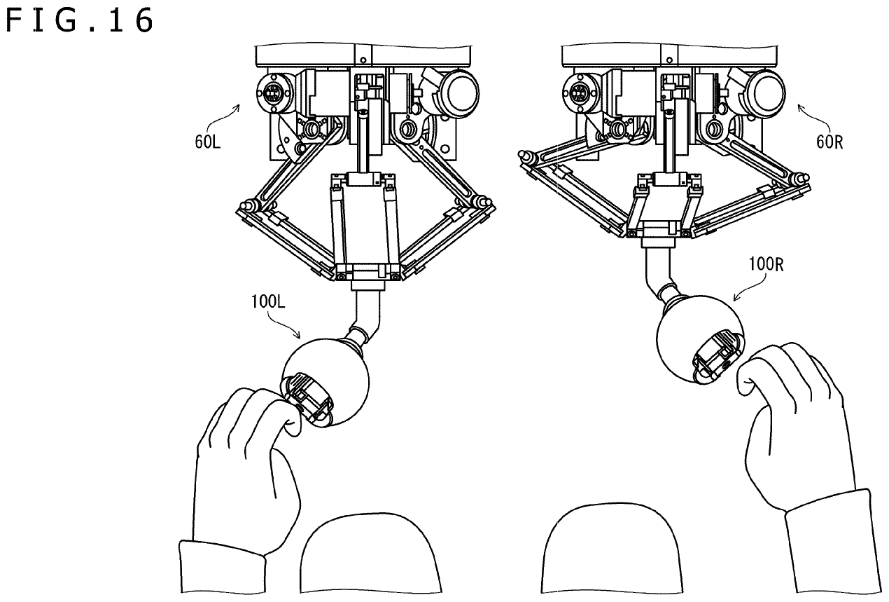

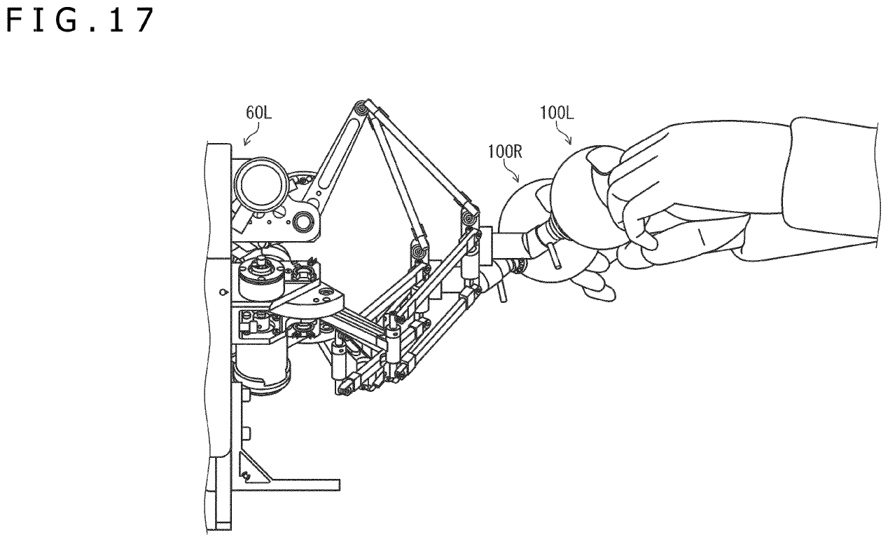

In general, humans are less likely to feel fatigued with the hands directed from top to bottom. FIGS. 16 and 17 each illustrate, as an example, a state in which operation sections 100L and 100R are attached to the distal end portions of the respective master apparatuses 60L and 60R with the opening sections for inserting the fingers facing upward, assuming that the hands are directed from top to bottom. It is to be noted that, however, FIG. 16 illustrates a state of the master apparatuses 60L and 60R viewed from above, while FIG. 17 illustrates a state of the master apparatuses 60L and 60R viewed from the side.

Further, with the elbows bent to a certain extent, a human can operate easily, respond instantly, and is less likely to hurt the user's elbows. Ergonomically, therefore, it can be said that a preferable attitude is such that the fingers (the thumbs and the index fingers or the middle fingers) of the user's left and right hands come from the left and right outer sides toward the front of the body, respectively. As can be seen from FIG. 16, therefore, the operation sections 100L and 100R are attached to the distal end portions of the respective master apparatuses 60L and 60R such that the opening sections for inserting the fingers face the left and right outer sides.

As illustrated in FIGS. 16 and 17, the operation sections 100L and 100R are attached to the distal end portions of the respective left and right master apparatuses 60L and 60R in consideration of ergonomics. Accordingly, the user can use a wide range of motion that the human arms originally have and can perform rotation input with a wide range of motion and three degrees of freedom. Conversely, if the operation sections 100L and 100R are attached in a direction in which human hands are difficult to direct without taking into account ergonomics (for example, in a case where the first opening section 111 and the second opening section 112 do not face the user facing the master apparatus 60), the user can use only a part of the range of motion of the user's arms to rotationally operate the operation sections 100L and 100R.

It is to be noted that although FIGS. 14 to 17 illustrate, as an example, the master apparatus 60 having the parallel link structure, it is, needless to say, possible to apply the three-axis translational structure other than the parallel link. For example, the main body of the master apparatus 60 may have another translational structure such as a structure in which linear actuators are serially connected. Further, the main body of the master apparatus 60 may have a translational structure with one or two axes, instead of three axes, depending on the application.

Further, although FIG. 2 illustrates, as an example, the wire 140 connected to the information input apparatus 100, a rope that connects the information input apparatus 100 to an outside fixed portion may be provided along the wire 140. The rope includes metal or a material that is difficult to be cut. In this way, the information input apparatus 100 can be prevented from being stolen or lost.

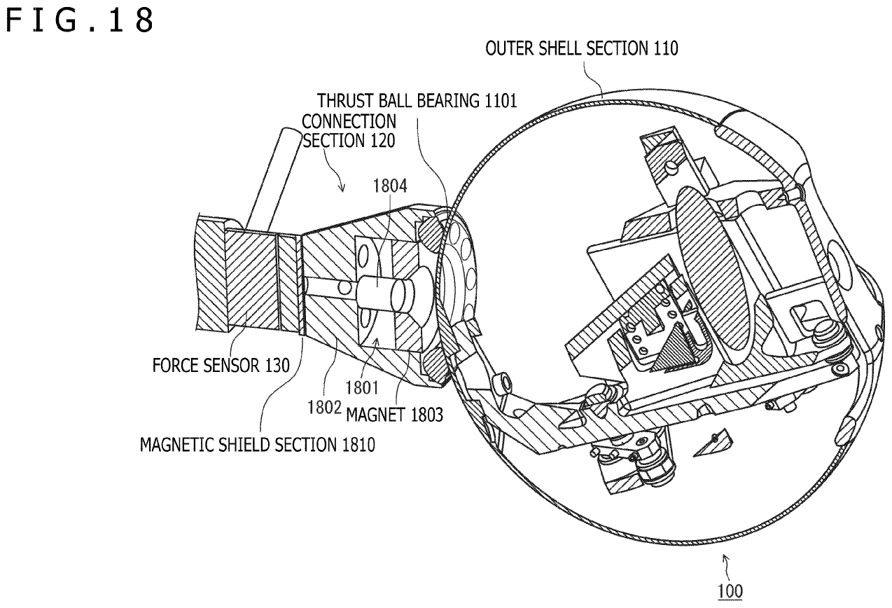

Further, FIG. 2 illustrates an example in which the connection section 120 includes the magnet and the contact surface 121 of the contact section 120 is subjected to low friction coating. In addition, FIG. 11 illustrates an example in which instead of the low friction coating, the thrust ball bearing 1101 is arranged to more smoothly rotate the outer shell section 110 about the three axes. In addition, as a modification of FIG. 17, FIG. 18 illustrates an example in which the position of the distance between the magnet of the connection section 120 and the outer shell section 110 is controllable.

In FIG. 18, the connection section 120 includes the thrust ball bearing 1101, a support section 1802, a magnet 1803, and a driving section 1804. The thrust ball bearing 1101 is arranged on the surface that is in contact with the outer shell section 110. The support section 1802 includes a hollow section 1801. The magnet 1803 is arranged in the hollow section 1801. The driving section 1804 displaces the position of the magnet 1803. The driving section 1804 drives the magnet 1803 such that the magnet 1803 moves back and forth in the radial direction of the outer shell section 110 inside the hollow section 1801.

The driving section 1804 can include any driving mechanism or an actuator element such as a ball screw, a link mechanism, or a linear actuator. It is to be noted that, however, the driving section 1804 preferably includes a ball screw from a viewpoint that driving using the ball screw is less likely to cause back driving. Further, although the driving section 1804 illustrated in FIG. 18 has a substantially cylindrical shape for the purpose of convenience, the driving section 1804 may have any shape as long as the driving section 1804 can be housed inside the hollow section 1801 and does not interfere with other members. It is to be noted that since the internal structure of the outer shell section 110 is similar to the internal structure of the outer shell section 110 described above, the detailed description is omitted here.

A controller not illustrated in FIG. 18 controls the driving of the driving section 1804. This controller may be the control apparatus 79, which controls force feedback and the like to the user. The controller can change the distance between the magnet 1803 and the outer shell section 110 by driving the driving section 1804 and controlling the position of the magnet 1803. Decreasing the distance between the magnet 1803 and the outer shell section 110 increases the force by which the magnet 1803 attracts the outer shell section 110 and makes it difficult for the user to rotationally operate the information input apparatus 100. On the other hand, increasing the distance between the magnet 1803 and the outer shell section 110 decreases the force by which the magnet 1803 attracts the outer shell section 110 and makes it easy for the user to rotationally operate the information input apparatus 100.

Therefore, when the user rotationally operates the information input apparatus 100, the control apparatus 79 can change the attractive force by which the magnet 1803 attracts the outer shell section 110 by controlling the driving of the driving section 1804 on the basis of the forces detected by the force sensors 91 on the slave apparatus 90 side. That is, the driving section 1804 controls the position of the magnet 1803. Accordingly, the resistance corresponding to the force acting on the surgical tool can be given and the force can be presented to the user.

It is to be noted that a magnetic shield section 1810 may be arranged between the connection section 120 and the force sensor 130 (or between the magnet 1803 and the force sensor 130) in order to prevent the influence of magnetism from exerting on the force sensor 130. For example, the magnetic shield section 1810 is manufactured with permalloy or the like.

Further, FIG. 18 illustrates the configuration in which a change in the relative positions of the magnet 1803 and the outer shell section 110 changes the magnetic force. Alternatively, the magnet 1803 is replaced with an electromagnet. Controlling the amount of current flowing through the electromagnet can change the magnetic force and realize the force presentation as above.

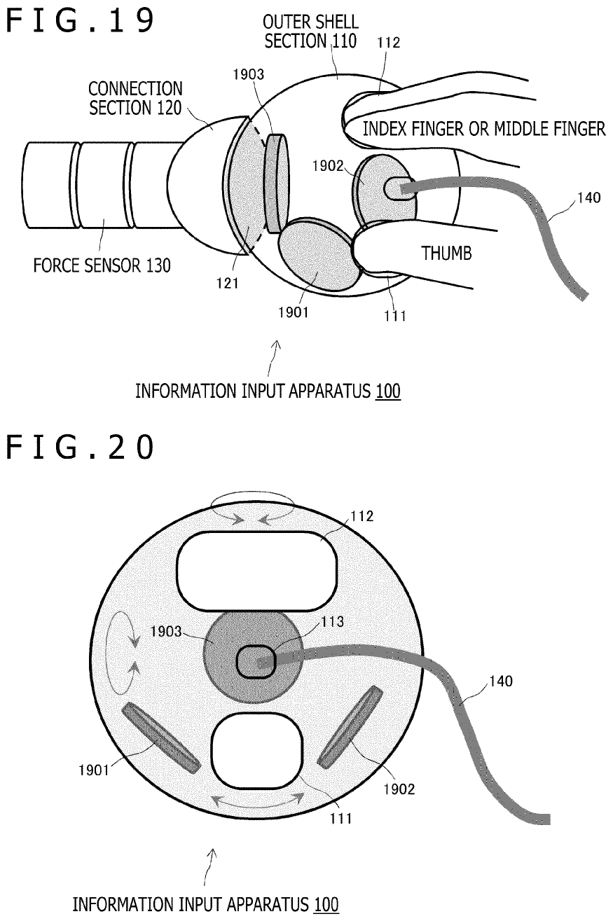

Further, FIGS. 19 and 20 illustrate an example of a configuration of the information input apparatus 100 in which actuators for presenting a rotational reaction force are arranged. It is to be noted that, however, FIG. 19 illustrates a state in which the information input apparatus 100 being operated by the user with the thumb, the index finger, and the middle finger is viewed from the side, while FIG. 20 illustrates a state of the information input apparatus 100 viewed from the front (the side including the first opening section 111 and the second opening section 112).

For example, at least two actuators are arranged inside the outer shell section 110 in order to present a rotational reaction force. In the example illustrated in FIGS. 19 and 20, three actuators 1901 to 1903 for presenting the rotational reaction force are arranged. Each of the actuators 1901 to 1903 may be an attitude control actuator such as a reaction wheel that is also used for an artificial satellite, a spacecraft, or the like, for example. Preferably, the actuators 1901 to 1903 are arranged in the vicinity of the position into which the thumb is inserted, such that the actuators 1901 to 1903 are plane-symmetrical to each other with respect to a plane passing through the opening sections 111 and 112 (that is, plane-symmetrical with respect to a plane that divides the information input apparatus 100 illustrated in FIG. 20 into left and right). Further, since the first opening section 111 into which only the thumb is inserted has a narrower hole width than the second opening section 112 into which the human element and the middle finger are inserted, the actuators 1901 and 1902 can be easily arranged. Further, the actuator 1903 at the center back (or on the opposite side of the wire hole 113) in FIG. 20 may be arranged on the slightly upper side for the weight balance.

The rotation of each of the actuators 1901 to 1903 is controlled by a controller that is not illustrated in FIGS. 19 and 20. This controller may be the control apparatus 79, which controls force feedback and the like to the user. The rotational reaction force can be presented to the user by rotating each of the actuators 1901 to 1903 on the basis of the control performed by the control apparatus 79. For example, the control apparatus 79 controls the rotation of each of the actuators 1901 to 1903 on the basis of the forces detected by the force sensors 91 on the slave apparatus 90 side. Accordingly, the forces detected by the slave apparatus 91 can be presented to the user.

Further, a wireless power feeding apparatus (not illustrated) may be provided in the connection section 120 (for example, in the hollow section 1801 included in the support section 1802 in FIG. 18) to wirelessly feed power to the information input apparatus 100. In this case, it is preferable that when the controller such as the control apparatus 79 detects that the information input apparatus 100 is not rotationally operated, wireless power feeding starts. This configuration can suppress a malfunction of the information input apparatus 100 due to wireless power feeding.

Further, whether the information input apparatus 100 is directly or indirectly connected to the connection section 120 may be detected, and the controller such as the control apparatus 79 may perform control based on the result of the detection. For example, a magnetic sensor provided in the connection section 120 or the information input apparatus 100 can detect a connection state between the connection section 120 and the information input apparatus 100 by detecting a magnetic change around the connection section 120. Then, the control apparatus 79 may present the connection state with the information input apparatus 100 to the user on the basis of the result of the detection. With such a configuration, in a case where the information input apparatus 100 has been directly or indirectly detached from the connection section 120 for a certain time, alert can be given to the user, or whether the connection state of the information input apparatus 100 is normal (for example, whether the information input apparatus 100 is arranged in a normal state directly or indirectly relative to the connection section 120) can be presented to the user.

Further, as a configuration that may be employed, whether or not the information input apparatus 100 is broken is determined on the basis of the magnetic sensor and the attitude position of the information input apparatus 100. Further, as a configuration that may be employed, whether an object on the ball attached to the connection section 120 is the genuine information input apparatus 100 is determined on the basis of the amount of magnetic change.

Further, the thrust ball bearing 1101 is preferably provided with a resin cover to suppress damage caused by friction. At this time, the controller such as the control apparatus 79 may detect the amount of friction generated by the resin cover and the sliding condition of the information input apparatus 100 (outer shell section 110) on the basis of the degree of rotation of the information input apparatus 100 or the like, and present the timing for replacement of the resin cover or timing for polishing the surface of the outer shell section 110 to the user on the basis of the result of the detection. Examples of the method for measuring the amount of friction and the sliding condition of the outer shell section 110 can include: a measuring method using the attitude change speed (rotational speed) when the information input apparatus 100 is moved by the self-weight thereof or a magnetic force while the user does not perform the input operation on the information input apparatus 100; and a measuring method using a temporal change in the attitude change speed (rotational speed) while the user is performing the input operation. It is to be noted that the detection of the temporal change is preferably performed by classification using machine learning.

The controller such as the control apparatus 79 can include an information processing apparatus including a circuit capable of realizing the functions described above. This type of information processing apparatus includes a CPU, a ROM (Read Only Memory), a RAM (Random Access Memory), a storage apparatus, and the like. For example, the functions of the controller such as the control apparatus 79 can be realized in the form that a program recorded in advance in the ROM or the storage apparatus is loaded into the RAM and is executed by the CPU. Further, the information processing apparatus may include hardware such as an FPGA (Field Programmable Gate Array) or an ASIC (Application Specific Integrated Circuit), or may include a GPU (Graphics Processing Unit). The hardware configuration of the information processing apparatus can be changed as appropriate according to the level of technology at the time of implementation of the present embodiment.

### INDUSTRIAL APPLICABILITY

The technology disclosed in the present specification has been described above in detail with reference to the specific embodiment. However, it is obvious that those skilled in the art can make modifications and substitutions of the embodiment without departing from the scope of the technology disclosed in the present specification.

In the present specification, the embodiment in which the technology disclosed in the present specification is applied to the master-slave type medical robot system has been mainly described. However, the scope of the technology disclosed in the present specification is not limited thereto. An input apparatus to which the technology disclosed in the present specification is applied can also be used for a master console of a robot system that is introduced into various industrial fields other than medical applications.

In addition, the input apparatus to which the technology disclosed in the present specification is applied can also be used for a game controller, an input device for a personal computer, the operation of a 3D model in CAD or the like, a six-axis input UI for operating an apparatus having a rotational structure such as a robot or a VR, and an input UI for operating the attitude of a camera mounted in a drone or a ceiling camera, for example.

In short, although the technology disclosed in the present specification has been described in the form of examples, the contents described in the present specification should not be interpreted in a limited manner. In order to determine the scope of the technology disclosed in the present specification, the claims should be taken into consideration.

It is to be noted that the technology disclosed in the present specification can also be configured as follows.

(1)

An information input apparatus including:

an outer shell section having a structure of a hollow sphere;

a connection section attracting and rotatably supporting the outer shell section; and

a rotation detection section configured to detect a rotation angle of the outer shell section.

(2)

The information input apparatus according to (1),

in which the rotation detection section is configured to detect rotation angles of three degrees of freedom of the outer shell section.

(3)

The information input apparatus according to any of (1) or (2),

in which the rotation detection section includes at least one or a combination of two or more of an acceleration sensor, an angle sensor, and a magnetic sensor.

(4)

The information input apparatus according to (3),

in which the rotation detection section is arranged in a vicinity of a center of the sphere.

(5)

The information input apparatus according to any of (1) or (2),

in which the rotation detection section includes a camera and is configured to detect the rotation angle of the outer shell section by tracking a pattern or a direction of a finger of a user operating the outer shell section with the pattern or the direction of the finger being imaged by the camera.

(6)

The information input apparatus according to any one of (1) to (5),

in which an outer diameter of the connection section is equal to or smaller than three-quarters of a diameter of the outer shell section.

(7)

The information input apparatus according to any one of (1) to (6),

in which a center of gravity of the outer shell section is positioned in a vicinity of a center of the sphere.

(8)

The information input apparatus according to any one of (1) to (7),

in which the outer shell section includes an opening section into which a finger of a user is inserted, and

the rotation detection section is configured to detect the rotation angle when the outer shell section is manually operated by the inserted finger.

(8-1)

The information input apparatus according to (8), further including:

a gripping mechanism arranged inside the outer shell section and configured to perform a pinch operation using the finger inserted from the opening section.

(9)

The information input apparatus according to (8), further including:

a finger detection sensor configured to detect that the finger has been inserted into the opening section.

(9-1)

The information input apparatus according to (9),

in which the finger detection sensor includes an optical sensor or an electrostatic sensor.

(10)

The information input apparatus according to any one of (1) to (8),

in which the outer shell section further includes:
a first opening section into which a first finger of a user is inserted; and
a second opening section into which a second finger of the user is inserted, and

a gripping mechanism arranged inside the outer shell section and configured to perform a pinch operation using the first finger and the second finger is further included.

(11)

The information input apparatus according to (10), further including:

an actuator configured to present a gripping force by causing the gripping mechanism to perform an opening/closing operation; and

an encoder configured to detect a rotation angle of the gripping mechanism gripped by the first finger and the second finger.

(12)

The information input apparatus according to any of (10) or (11),

in which the gripping mechanism is arranged such that a center between the first finger and the second finger gripping the gripping mechanism is positioned in a vicinity of a center of the sphere.

(13)

The information input apparatus according to (11),

in which a center of gravity of the outer shell section including the actuator is positioned in a vicinity of a center of the sphere.

(14)

The information input apparatus according to any one of (1) to (13),

in which the outer shell section includes a plurality of faces coupled to each other and including a first outer shell spherical section and a second outer shell spherical section.

(15)

The information input apparatus according to any one of (1) to (14),

in which a surface of at least one of the outer shell section or the connection section is subjected to a treatment for reducing friction.

(16)

The information input apparatus according to any one of (1) to (15),

in which the connection section attracts the outer shell section by any of a magnetic force of a magnet, an air pressure, and an electrostatic force.

(17)

The information input apparatus according to any one of (1) to (16), further including:

a force sensor configured to detect an external force acting on the outer shell section.

(18)

The information input apparatus according to any one of (10) to (13),

in which the outer shell section includes: one or more internal parts; and a wire hole into which a wire for electrically connecting the one or more internal parts to an outside is inserted, the wire hole lying between the first opening section and the second opening section.

(19)

The information input apparatus according to any one of (1) to (18),

in which the information input apparatus is attached to a master apparatus in a master-slave system and used as an operation section configured to be operated by a user.

(20)

A master-slave type medical system,

in which a master apparatus includes:
an operation section including
   an outer shell section having a structure of a hollow sphere, and
   a rotation detection section configured to detect a rotation angle of the outer shell section; and
a translational structure section attracting and rotatably supporting the outer shell section of the operation section and configured to detect a translation force acting on the outer shell section or present the translation force.
   (20-1)

The medical system according to (20),

in which the translational structure section has a parallel link structure including a plurality of links each supporting the outer shell section at a distal end side and being turnably supported by a main body of the master apparatus at a proximal end side, and

an actuator configured to rotationally drive each of the plurality of links and present the translation force is further included.

(21)

The medical system according to (20), further including:

a slave apparatus including a medical instrument and configured to control an operation of the medical instrument on the basis of a detection signal in the operation section or the translational structure section in the master apparatus.

### REFERENCE SIGNS LIST

1 . . . Robot system
20 . . . Support arm section, 20a to 20c . . . Link section
21 . . . Driving link, 22 . . . Passive link
30 . . . Main body section
60 . . . Master apparatus
61 . . . Force sensor, 63 . . . Rotation angle sensor
65 . . . Motor, 65a to 65c . . . Motor
67 . . . Vibration generating source, 69 . . . Speaker
70 . . . First vibration transmitting section
71 . . . Amplifier, 73 . . . Frequency characteristic correction circuit
75 . . . Band-pass filter, 77 . . . Driving circuit
79 . . . Control apparatus
80 . . . Second vibration transmitting section
81 . . . Amplifier, 83 . . . Frequency characteristic correction circuit
85 . . . Band-pass filter, 87 . . . Driving circuit
90 . . . Slave apparatus
91 . . . Force sensor, 93 . . . Rotation angle sensor, 95 . . . Motor
97 . . . Tactile vibration sensor, 99 . . . Auditory vibration sensor
100 . . . Information input apparatus
110 . . . Outer shell section
111 . . . First opening section, 112 . . . Second opening section, 113 . . . Wire hole
120 . . . Connection section, 121 . . . Contact surface
130 . . . Force sensor, 140 . . . Wire
501 . . . Outer shell front section, 502 . . . Outer shell rear section
601 . . . Rotation angle sensor (IMU), 602 . . . Substrate section
611 . . . First gripping plate, 612 . . . Second gripping plate, 613 . . . Finger pad recess
621 . . . Motor
631, 632 . . . Finger detection sensor
641, 642 . . . General-purpose switch
651 . . . Wire fixing frame, 661 . . . Spherical assembling section
701 . . . Fixed link, 702 . . . Driving link
703 . . . Driven link, 704 . . . Intermediate link
706 . . . First transmission link, 707 . . . Second transmission link
1101 . . . Thrust ball bearing
1801 . . . Hollow section, 1802 . . . Support section, 1803 . . . Magnet
1804 . . . Driving section, 1810 . . . Magnetic shield section
1901 to 1903 . . . Actuator (reaction wheel)

1. An information input apparatus comprising:
an outer shell section having a structure of a hollow sphere;
a connection section attracting and rotatably supporting the outer shell section; and
a rotation detection section configured to detect a rotation angle of the outer shell section.

2. The information input apparatus according to claim 1,
wherein the rotation detection section is configured to detect rotation angles of three degrees of freedom of the outer shell section.

3. The information input apparatus according to claim 1,
wherein the rotation detection section includes at least one or a combination of two or more of an acceleration sensor, an angle sensor, and a magnetic sensor.

4. The information input apparatus according to claim 3,
wherein the rotation detection section is arranged in a vicinity of a center of the sphere.

5. The information input apparatus according to claim 1,
wherein the rotation detection section includes a camera and is configured to detect the rotation angle of the outer shell section by tracking a pattern or a direction of a finger of a user operating the outer shell section with the pattern or the direction of the finger being imaged by the camera.

6. The information input apparatus according to claim 1,
wherein an outer diameter of the connection section is equal to or smaller than three-quarters of a diameter of the outer shell section.

7. The information input apparatus according to claim 1,
wherein a center of gravity of the outer shell section is positioned in a vicinity of a center of the sphere.

8. The information input apparatus according to claim 1,
wherein the outer shell section includes an opening section into which a finger of a user is inserted, and
the rotation detection section is configured to detect the rotation angle when the outer shell section is manually operated by the inserted finger.

9. The information input apparatus according to claim 8, further comprising:
a finger detection sensor configured to detect that the finger has been inserted into the opening section.

10. The information input apparatus according to claim 1,
wherein the outer shell section further includes:
a first opening section into which a first finger of a user is inserted; and
a second opening section into which a second finger of the user is inserted, and
a gripping mechanism arranged inside the outer shell section and configured to perform a pinch operation using the first finger and the second finger is further included.

11. The information input apparatus according to claim 10, further comprising:
an actuator configured to present a gripping force by causing the gripping mechanism to perform an opening/closing operation; and
an encoder configured to detect a rotation angle of the gripping mechanism gripped by the first finger and the second finger.

12. The information input apparatus according to claim 10,
wherein the gripping mechanism is arranged such that a center between the first finger and the second finger gripping the gripping mechanism is positioned in a vicinity of a center of the sphere.

13. The information input apparatus according to claim 11,
wherein a center of gravity of the outer shell section including the actuator is positioned in a vicinity of a center of the sphere.

14. The information input apparatus according to claim 1,
wherein the outer shell section includes a plurality of faces coupled to each other and including a first outer shell spherical section and a second outer shell spherical section.

15. The information input apparatus according to claim 1,
wherein a surface of at least one of the outer shell section or the connection section is subjected to a treatment for reducing friction.

16. The information input apparatus according to claim 1,
wherein the connection section attracts the outer shell section by any of a magnetic force of a magnet, an air pressure, and an electrostatic force.

17. The information input apparatus according to claim 1, further comprising:
a force sensor configured to detect an external force acting on the outer shell section.

18. The information input apparatus according to claim 10,
wherein the outer shell section includes: one or more internal parts; and a wire hole into which a wire for electrically connecting the one or more internal parts to an outside is inserted, the wire hole lying between the first opening section and the second opening section.

19. The information input apparatus according to claim 1,
wherein the information input apparatus is attached to a master apparatus in a master-slave system and used as an operation section configured to be operated by a user.

20. A master-slave type medical system,
wherein a master apparatus includes:
an operation section including
an outer shell section having a structure of a hollow sphere, and
a rotation detection section configured to detect a rotation angle of the outer shell section; and
a translational structure section attracting and rotatably supporting the outer shell section of the operation section and configured to detect a translation force acting on the outer shell section or present the translation force.

21. The medical system according to claim 20, further comprising:
a slave apparatus including a medical instrument and configured to control an operation of the medical instrument on a basis of a detection signal in the operation section or the translational structure section in the master apparatus.
